(19) United States
(12) Patent Application Publication
(10) Pub. No.: US 2006/0248603 A1
(43) Pub. Date: Nov. 2, 2006

(54) Novel fibrillin-like polypeptides

(75) Inventors: Gregg McAllister, Charlestown, MA (US); Jadwiga Bienkowska, Cambridge, MA (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Applied Research Systems ARS Holding N.V., Curacao (NL)

(21) Appl. No.: 10/540,847

(22) PCT Filed: Dec. 22, 2003
(86) PCT No.: PCT/EP03/51089

§ 371 Date: Apr. 21, 2006

Related U.S. Application Data
(60) Provisional application No. 60/436,835, filed on Dec. 27, 2002.

(51) Int. Cl. A01K 67/027; C07H 21/04; C12P 21/06; C12N 9/64; C07K 16/40

(52) U.S. Cl. 800/8; 530/388.26; 435/226; 435/69.1; 435/354; 536/23.2; 435/320.1

(57) ABSTRACT

The present invention discloses open reading frames (ORFS) in human genome encoding for novel fibrillin-like polypeptides, and reagents related thereto including variants, mutants and fragments of said polypeptides, as well as ligands and antagonists directed against them. The invention provides methods for identifying and making these molecules, for preparing pharmaceutical compositions containing them, and for using them in the diagnosis, prevention and treatment of diseases.

### FIELD OF THE INVENTION

The present invention relates to nucleic acid sequences identified in the human genome as encoding for novel polypeptides, more specifically for fibrillin-like polypeptides.

All publications, patents and patent applications cited herein are incorporated in full by reference.

### BACKGROUND OF THE INVENTION

Many novel polypeptides have been already identified by applying strict homology criteria to known polypeptides of the same family. However, since the actual content in polypeptide-encoding sequences in the human genome for fibrillin-like polypeptides (and for any other protein family) is still unknown, the possibility still exists to identify DNA sequence encoding polypeptide having fibrillin-like polypeptide activities by applying alternative and less strict homology/structural criteria to the totality of Open Reading Frames (ORFs, that is, genomic sequences containing consecutive triplets of nucleotides coding for amino acids, not interrupted by a termination codon and potentially translatable in a polypeptide) present in the human genome.

The ability for cells to make and secrete extracellular proteins is central to many biological processes. Enzymes, growth factors, extracellular matrix proteins and signalling molecules are all secreted by cells. This is through fusion of a secretory vesicle with the plasma membrane. In most cases, but not all, proteins are directed to the endoplasmic reticulum and into secretory vesicles by a signal peptide. Signal peptides are cis-acting sequences that affect the transport of polypeptide chains from the cytoplasm to a membrane bound compartment such as a secretory vesicle. Polypeptides that are targeted to the secretory vesicles are either secreted into the extracellular matrix or are retained in the plasma membrane. The polypeptides that are retained in the plasma membrane will have one or more transmembrane domains. Examples of secreted proteins that play a central role in the functioning of a cell are cytokines, hormones, extracellular matrix proteins (adhesion molecules), proteases, and growth and differentiation factors.

Microfibril bundles and proteins found in association with these bundles, particularly attachment molecules, are of interest in the field of dermatology, particularly in the study of skin which has been damaged from aging, injuries or the sun. Fibrillin microfibrils define the continuous elastic network of skin, and are present in dermis as microfibril bundles devoid of measurable elastin extending from the dermal-epithelial junction and as components of the thick elastic fibres present in the deep reticular dermis.

Fibrillin-1 is a modular glycoprotein with amino-terminal anaphlatoxin-like modules followed by nine epidermal growth factor (EGF)-like modules and, depending on alternative splicing, four possible carboxyl termini. Fibrillin-2 is a novel extracellular matrix protein frequently found in close association with microfibrils containing either fibronectin or fibulin. Thus, fibrillin, and molecules related thereto are of interest, particularly for the use of preventing skin from being damaged from aging, injuries or the sun, or for restoring skin damaged from same. Moreover, these molecules are generally of interest in the study of connective tissue and attachment molecules and related mechanisms. Fibrillin, and related molecules are further described in Adams, et al., J. Mol. Biol., 272(2):226-36 (1997); Kielty and Shuttleworth, Microsc. Res. Tech., 38(4):413-27 (1997); and Child, J. Card. Surg,. 12(2Supp.):131-3 (1997).

Thus, fibrillin, and molecules related thereto are of significant interest, particularly for the use of preventing skin from being damaged from aging, injuries or the sun, or for restoring skin damaged from same. Moreover, these molecules are generally of interest in the study of connective tissue and attachment molecules and related mechanisms.

The exact roles that fibrillins have in the formation and/or stabilization of extracellular matrix structures as well as their effects on cellular behavior are currently under investigation. However, it is clear that the identification of novel fibrillin-like proteins is of significant importance in increasing understanding of the underlying pathways that lead to certain disease states in which these proteins are implicated, and in developing more effective gene or drug therapies to treat these disorders.

### SUMMARY OF THE INVENTION

The invention is based upon the identification of an Open Reading Frame (ORF) in the human genome encoding a novel fibrillin-like polypeptide. This polypeptide will be referred to herein as the SCS0008 polypeptide. Based on the full coding sequence of the SCS0008 prediction, two splice variants of SCS0008, SCS0008 -SV1 and SCS0008-SV2 were additionally discovered. SCS0008, SCS0008-SV1 and SCS0008-SV2 will be referred herein as SCS0008.

Accordingly, the invention provides isolated SCS0008 polypeptides having the amino acid sequence given by SEQ ID NO: 2, SEQ ID NO: 3, SEQ ID NO: 4, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, and their mature forms, histidine forms, variants, and fragments, as polypeptides having the activity of fibrillin-like polypeptides. The invention includes also the nucleic acids encoding them, vectors containing such nucleic acids, and cell containing these vectors or nucleic acids, as well as other related reagents such as fusion proteins, ligands, and antagonists.

The invention provides methods for identifying and making these molecules, for preparing pharmaceutical compositions containing them, and for using them in the diagnosis, prevention and treatment of diseases.

### BRIEF DESCRIPTION OF THE FIGURES

FIG. 1: alignment of the SCS0008 ORF with known related polypeptide sequences.

FIG. 2: Clustal W alignment of SCS0008 predicted amino acid sequence with deduced amino acid sequences for cloned splice variants SCS0008SV1 and SCS0008SV2.

FIG. 3: Nucleotide sequence of SCS0008 prediction with translation.

FIG. 4: Nucleotide sequence with translation of SCS0008 product cloned using primers SCS0008-CP1nest and SCS0008-CP2nest.

FIG. 5: Map of pCR4-TOPO-SCS0008SV1.

FIG. 6: Map of expression vector pEAK12d.

FIG. 7: Map of pDONR 221.

FIG. 8: Map of Expression vector pDEST12.2.

FIG. 9: Map of pENTR-SCS0008SV1-6HIS.

FIG. 10: Map of pEAK12d-SCS0008SV1-6HIS.

FIG. 11: Map of pDEST12.2-SCS0008SV1-6HIS.

FIG. 12: Nucleotide sequence of SCS0008 prediction with translation.

FIG. 13: Nucleotide sequence with translation of SCS0008 product cloned using primers SCS0008-CP1 nest and SCS0008-CP2nest.

FIG. 14: Map of pCR4-TOPO-SCS0008-SV2.

FIG. 15: Map of expression vector pEAK12d.

FIG. 16: Map of Expression vector pDEST12.2.

FIG. 17: Map of pDONR 221.

FIG. 18: Map of pENTR-SCS0008SV2-6HIS.

FIG. 19: Map of pEAK12d-SCS0008SV2-6HIS.

FIG. 20: Map of pDEST12.2-SCS0008SV2-6HIS.

FIG. 21: SMART Domains alignment of SCS0008, SCS0008-SV1, SCS0008-SV2, mouse nephronectin, and other related sequences.

### DETAILED DESCRIPTION OF THE INVENTION

According to a first aspect of the present invention there is provided an isolated polypeptide having fibrillin-like activity selected from the group consisting of:
a) the amino acid sequences as recited in SEQ ID NO: 2; SEQ ID NO: 6, SEQ ID NO: 7, SEQ ID NO: 13, SEQ ID NO: 14;
b) the mature form of the polypeptides whose sequences are recited in SEQ ID NO: 2 (SEQ ID NO:3), SEQ ID NO: 6 (SEQ ID NO:8), SEQ ID NO: 7 (SEQ ID NO: 9), SEQ ID NO: 13 (SEQ ID NO: 15), SEQ ID NO: 14 (SEQ ID NO: 16);
c) the histidine tag form of the polypeptides whose sequences are recited in SEQ ID NO: 2 (SEQ ID NO:4), SEQ ID NO: 6 (SEQ ID NO:10), SEQ ID NO: 7 (SEQ ID NO: 11), SEQ ID NO: 13 (SEQ ID NO: 17), SEQ ID NO: 14 (SEQ ID NO: 18);
d) a variant of the amino acid sequence recited in SEQ ID NO: 2, SEQ ID NO: 6, SEQ ID NO: 7, SEQ ID NO:-13, SEQ ID NO: 14, wherein any amino acid specified in the chosen sequence is non-conservatively substituted, provided that no more than 15% of the amino acid residues in the sequence are so changed;
e) an active fragment, precursor, salt, or derivative of the amino acid sequences given in a) to d).

The novel SCS0008 polypeptide described herein was identified on the basis of proprietary bioinformatics techniques. EGF domains were used as query sequences for searches of databases and the final annotation was attributed on the basis of amino acid sequence homology.

The totality of amino acid sequences obtained by translating the known ORFs in the human genome were challenged using this consensus sequence, and the positive hits were further screened for the presence of predicted specific structural and functional “signatures” that are distinctive of a polypeptide of this nature, and finally selected by comparing sequence features with known fibrillin-like polypeptides. Therefore, the novel polypeptides of the invention can be predicted to have fibrillin-like activities.

The terms “active” and “activity” refer to the fibrillin-like properties predicted for the fibrillin-like polypeptide whose amino acid sequence is presented in SEQ ID NO: 2, SEQ ID NO: 3, SEQ ID NO: 4, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, in the present application. These properties include the ability to form microfibril bundles in connective tissue.

In a second aspect, the invention provides a purified nucleic acid molecule which encodes a polypeptide of the first aspect of the invention. Preferably, the purified nucleic acid molecule has the nucleic acid sequence as recited in SEQ ID NO:1 (encoding the fibrillin-like polypeptide whose amino acid sequence is recited in SEQ ID NO:2), SEQ ID NO:5 (encoding the amino acid sequence recited in SEQ ID NO:6), or SEQ ID NO:12 (encoding the amino acid sequence recited in SEQ ID NO:13).

In a third aspect, the invention provides a purified nucleic acid molecule which hydridizes under high stringency conditions with a nucleic acid molecule of the second aspect of the invention.

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention.

In a sixth aspect, the invention provides a ligand which binds specifically to, and which preferably inhibits the fibrillin-like activity of a polypeptide of the first aspect of the invention. Ligands to a polypeptide according to the invention may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional mimetcs of the aforementioned.

In a seventh aspect, the invention provides a compound that is effective to alter the expression of a natural gene which encodes a polypeptide of the first aspect of the invention or to regulate the activity of a polypeptide of the first aspect of the invention.

A compound of the seventh aspect of the invention may either increase (agonise) or decrease (antagonise) the level of expression of the gene or the activity of the polypeptide. Importantly, the identification of the function of the fibrillin-like polypeptide of the invention allows for the design of screening methods capable of identifying compounds that are effective in the treatment and/or diagnosis of disease.

In an eighth aspect, the invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy or diagnosis. These molecules may also be used in the manufacture of a medicament for the treatment of diseases such as those in which skin is damaged from aging, injuries or the sun, or for restoring skin damaged from the same, also multiple sclerosis, cancer, bone, joint or ligament reconstruction after fractures or lesions, osteoarthritis, rheumatoid arthritis, osteoporosis, cardiovascular diseases and fibrosis (including liver fibrosis, kidney fibrosis, hepatitis and renal disorders) as well as those conditions and disorders mentioned in the therapeutic uses below.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patent and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect, the invention provides for the use of a polypeptide of the first aspect of the invention as a fibrillin-like protein. Suitable uses include use as a secreted glycoprotein, in particular in the context of tissue repair and remodeling, as a result of the ability of the protein to bind to extracellular matrix structures such as connective tissue fibers, basement membranes and blood clots through interacting with other extracellular matrix proteins such as fibronectin, laminins, nidogen, perlecan, fibulin and fibrinogen.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in the manufacture of a medicament for the diagnosis or treatment of a disease, such as those in which skin is damaged from aging, injuries or the sun, or for restoring skin damaged from the same, also multiple sclerosis, cancer, bone, joint or ligament reconstruction after fractures or lesions, osteoarthritis, rheumatoid arthritis, osteoporosis, cardiovascular diseases and fibrosis (including liver fibrosis, kidney fibrosis, hepatitis and renal disorders) as well as those conditions and disorders mentioned in the therapeutic uses below.

In a thirteenth aspect, the invention provides a method of treating a disease in a patent comprising administering to the patient a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be used in screening regimes for the identification of compounds that are effective in the treatment or diagnosis of such a disease.

A summary of standard techniques and procedures which may be employed in order to utilize the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of the those working in the art Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

The first aspect of the invention includes variants of the amino acid sequence recited in SEQ ID NO: 2, SEQ ID NO: 3, SEQ ID NO: 4, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17 and SEQ ID NO: 18, wherein any amino acid specified in the chosen sequence is non-conservatively substituted, provided that no more than 15% of the amino acid residues in the sequence are so changed. Protein sequences having the indicated number of non-conservative substitutions can be identified using commonly available bioinformatic tools (Mulder N J and Apweiler R, 2002; Rehm B H, 2001).

In addition to such sequences, a series of polypeptides forms part of the disclosure of the invention. Being fibrillin-like polypeptides known to go through maturation processes including the proteolytic removal of N-terminal sequences (by signal peptidases and other proteolytic enzymes), the present application also claims the mature form of the polypeptide whose sequences are recited in SEQ ID NO: 2 (SEQ ID NO:3), SEQ ID NO: 6 (SEQ ID NO:8), SEQ ID NO: 7 (SEQ ID NO: 9), SEQ ID NO: 13 (SEQ ID NO: 15), SEQ ID NO: 14 (SEQ ID NO: 16). Mature forms are intended to include any polypeptide showing fibrillin-like activity and resulting from in vivo (by the expressing cells or animals) or in vitro (by modifying the purified polypeptides with specific enzymes) post-translational maturation processes. Other alternative mature forms can also result from the addition of chemical groups such as sugars or phosphates. The present application also claims the histidine tag form of the polypeptide whose sequences are recited in SEQ ID NO: 2 (SEQ ID NO:4), SEQ ID NO: 6 (SEQ ID NO:10), SEQ ID NO: 7 (SEQ ID NO: 11), SEQ ID NO: 13 (SEQ ID NO: 17), SEQ ID NO: 14 (SEQ ID NO: 18).

Other claimed polypeptides are the active variants of the amino acid sequences given by SEQ ID NO: 2, SEQ ID NO: 3, SEQ ID NO: 4, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17 or SEQ ID NO: 18, wherein any amino acid specified in the chosen sequence is non-conservatively substituted, provided that no more than 15%, preferably no more that 10%, 5%, 3%, or 1%, of the amino acid residues in the sequence are so changed. The indicated percentage has to be measured over the novel amino acid sequences disclosed.

In accordance with the present invention, any substitution should be preferably a “conservative” or “safe” substitution, which is commonly defined a substitution introducing an amino acids having sufficiently similar chemical properties (eg a basic, positively charged amino acid should be replaced by another basic, positively charged amino acid), in order to preserve the structure and the biological function of the molecule.

The literature provide many models on which the selection of conservative amino acids substitutions can be performed on the basis of statistical and physico-chemical studies on the sequence and/or the structure of proteins (Rogov S I and Nekrasov A N, 2001). Protein design experiments have shown that the use of specific subsets of amino acids can produce foldable and active proteins, helping in the classification of amino acid “synonymous” substitutions which can be more easily accommodated in protein structure, and which can be used to detect functional and structural homologs and paralogs (Murphy L R et al., 2000). The groups of synonymous amino acids and the groups of more preferred synonymous amino acids are shown in Table I.

Active variants having comparable, or even improved, activity with respect of corresponding fibrillin-like polypeptides may result from conventional mutagenesis technique of the encoding DNA, from combinatorial technologies at the level of encoding DNA sequence (such as DNA shuffling, phage display/selection), or from computer-aided design studies, followed by the validation for the desired activities as described in the prior art.

Specific, non-conservative mutations can be also introduced in the polypeptides of the invention with different purposes. Mutations reducing the affinity of the fibrillin-like polypeptide may increase its ability to be reused and recycled, potentially increasing its therapeutic potency (Robinson C R, 2002). Immunogenic epitopes eventually present in the polypeptides of the invention can be exploited for developing vaccines (Stevanovic S, 2002), or eliminated by modifying their sequence following known methods for selecting mutations for increasing protein stability, and correcting them (van den Burg B and Eijsink V, 2002; WO 02/05146, WO 00/34317, WO 98/52976).

Further alternative polypeptides of the invention are active fragments, precursors, salts, or functionally-equivalent derivatives of the amino acid sequences described above.

Fragments should present deletions of terminal or internal amino acids not altering their function, and should involve generally a few amino acids, e.g., under ten, and preferably under three, without removing or displacing amino acids which are critical to the functional conformation of the proteins. Small fragments may form an antigenic determinant.

The “precursors” are compounds which can be converted into the compounds of present invention by metabolic and enzymatic processing prior or after the administration to the cells or to the body.

The term “salts” herein refers to both salts of carboxyl groups and to acid addition salts of amino groups of the polypeptides of the present invention. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for exam pie, salts with mineral acids such as, for example, hydrochloric acid or sulfuric acid, and salts with organic acids such as, for example, acetic acid or oxalic acid. Any of such salts should have substantially similar activity to the peptides and polypeptides of the invention or their analogs.

The term “derivatives” as herein used refers to derivatives which can be prepared from the functional groups present on the lateral chains of the amino acid moieties or on the amino- or carboxy-terminal groups according to known methods. Such molecules can result also from other modifications which do not normally alter primary sequence, for example in vivo or in vitro chemical dirivativization of polypeptides (acetylation or carboxylation), those made by modifying the pattern of phosphorylation (introduction of phosphotyrosine, phosphoserine, or phosphothreonine residues) or glycosylation (by exposing the polypeptide to mammalian glycosylating enzymes) of a peptide during its synthesis and processing or in further processing steps. Alternatively, derivatives may include esters or aliphatic amides of the carboxyl-groups and N-acyl derivatives of free amino groups or O-acyl derivatives of free hydroxyl-groups and are formed with acyl-groups as for example alcanoyl- or aryl-groups.

The generation of the derivatives may involve a site-directed modification of an appropriate residue, in an internal or terminal position. The residues used for attachment should they have a side-chain amenable for polymer attachment (i.e., the side chain of an amino acid bearing a functional group, e.g., lysine, aspartic acid, glutamic acid, cysteine, histidine, etc.). Alternatively, a residue having a side chain amenable for polymer attachment can replace an amino acid of the polypeptide, or can be added in an internal or terminal position of the polypeptide. Also, the side chains of the genetically encoded amino acids can be chemically modified for polymer attachment, or unnatural amino acids with appropriate side chain functional groups can be employed. The preferred method of attachment employs a combination of peptide synthesis and chemical ligation. Advantageously, the attachment of a water-soluble polymer will be through a biodegradable linker, especially at the amino-terminal region of a protein. Such modification acts to provide the protein in a precursor (or “pro-drug”) form, that, upon degradation of the linker releases the protein without polymer modification.

Polymer attachment may be not only to the side chain of the amino acid naturally occurring in a specific position of the antagonist or to the side chain of a natural or unnatural amino acid that replaces the amino acid naturally occurring in a specific position of the antagonist, but also to a carbohydrate or other moiety that is attached to the side chain of the amino acid at the target position. Rare or unnatural amino acids can be also introduced by expressing the protein in specifically engineered bacterial strains (Bock A, 2001).

All the above indicated variants can be natural, being identified in organisms other than humans, or artificial, being prepared by chemical synthesis, by site-directed mutagenesis techniques, or any other known technique suitable thereof, which provide a finite set of substantially corresponding mutated or shortened peptides or polypeptides which can be routinely obtained and tested by one of ordinary skill in the art using the teachings presented in the prior art.

The novel amino acid sequences disclosed in the present patent application can be used to provide different kind of reagents and molecules. Examples of these compounds are binding proteins or antibodies that can be identified using their full sequence or specific fragments, such as antigenic determinants. Peptide libraries can be used in known methods (Tribbick G, 2002) for screening and characterizing antibodies or other proteins binding the claimed amino acid sequences, and for identifying alternative forms of the polypeptides of the invention having similar binding properties.

The present patent application discloses also fusion proteins comprising any of the polypeptides described above. These polypeptides should contain protein sequence heterologous to the one disclosed in the present patent application, without significantly impairing the fibrillin-like activity of the polypeptide and possibly providing additional properties. Examples of such properties are an easier purification procedure, a longer lasting half-life in body fluids, an additional binding moiety, the maturation by means of an endoproteolytic digestion, or extracellular localization. This latter feature is of particular importance for defining a specific group of fusion or chimeric proteins included in the above definition since it allows the claimed molecules to be localized in the space where not only isolation and purification of these polypeptides is facilitated, but also where generally fibrillin-like polypeptides and their receptor interact Design of the moieties, ligands, and linkers, as well methods and strategies for the construction, purification, detection and use of fusion proteins are disclosed in the literature (Nilsson J et al., 1997; Methods Enzymol, Vol. 326-328, Academic Press, 2000). The preferred one or more protein sequences which can be comprised in the fusion proteins belong to these protein sequences: membrane-bound protein, immunoglobulin constant region, multimerization domains, extracellular proteins, signal peptide-containing proteins, export signal-containing proteins. Features of these sequences and their specific uses are disclosed in a detailed manner, for example, for albumin fusion proteins (WO 01/77137), fusion proteins including multimerization domain (WO 01102440, WO 00/24782), immunoconjugates (Garnett M C, 2001), or fusion protein providing additional sequences which can be used for purifying the recombinant products by affinity chromatography (Constans A, 2002; Burgess R R and Thompson N E, 2002; Lowe C R et al., 2001; J. Bioch. Biophy. Meth., vol. 49 (1-3), 2001; Sheibani N, 1999).

The polypeptides of the invention can be used to generate and characterize ligands binding specifically to them. These molecules can be natural or artificial, very different from the chemical point of view (binding proteins, antibodies, molecularly imprinted polymers), and can be produced by applying the teachings In the art (WO 02/74938; Kuroiwa Y et al., 2002; Haupt K, 2002; van Dijk M A and van de Winkel J G, 2001; Gavilondo J V and Larrick J W, 2000). Such ligands can antagonize or inhibit the fibrillin-like activity of the polypeptide against which they have been generated. In particular, common and efficient ligands are represented by extracellular domain of a membrane-bound protein or antibodies, which can be in the form monoclonal, polyclonal, humanized antibody, or an antigen binding fragment.

The polypeptides and the polypeptide-based derived reagents described above can be in alternative forms, according to the desired method of use and/or production, such as active conjugates or complexes with a molecule chosen amongst radioactive labels, fluorescent labels, biotin, or cytotoxic agents.

Specific molecules, such as peptide mimetics, can be also designed on the sequence and/or the structure of a polypeptide of the invention. Peptide mimetics (also called peptidomimetics) are peptides chemically modified at the level of amino acid side chains, of amino acid chirality, and/or of the peptide backbone. These alterations are intended to provide agonists or antagonists of the polypeptides of the invention with improved preparation, potency and/or pharmacokinetics features.

For example, when the peptide is susceptible to cleavage by peptidases following injection into the subject is a problem, replacement of a particularly sensitive peptide bond with a non-cleavable peptide mimetic can provide a peptide more stable and thus more useful as a therapeutic. Similarly, the replacement of an L-amino acid residue is a standard way of rendering the peptide less sensitive to proteolysis, and finally more similar to organic compounds other than peptides. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4-dinitrophenyl. Many other modifications providing increased potency, prolonged activity, easiness of purification, and/or increased half-life are disclosed in the prior art (WO 02/10195; Villain M et al., 2001).

Preferred alternative, synonymous groups for amino acids derivatives included in peptide mimetics are those defined in Table II. A non-exhaustive list of amino acid derivatives also include aminoisobutyric acid (Aib), hydroxyproline (Hyp), 1,2,3,4-tetrahydro-isoquinoline-3-COOH, in doline-2carboxylic acid, 4-difluoro-proline, L-thiazolidine-4-carboxylic acid, L-homoproline, 3,4-dehydro-proline, 3,4-dihydroxy-phenylalanine, cyclohexyl-glycine, and phenylglycine.

By “amino acid derivative” is intended an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem AG, Switzerland; Bachem, USA).

Various methodologies for incorporating unnatural amino acids derivatives into proteins, using both in vitro and in vivo translation systems, to probe and/or improve protein structure and function are disclosed in the literature (Dougherty D A, 2000). Techniques for the synthesis and the development of peptide mimetics, as well as non-peptide mimetics, are also well known in the art (Golebiowski A et al., 2001; Hruby V J and Balse P M, 2000; Sawyer T K, in “Structure Based Drug Design”, edited by Veerapandian P, Marcel Dekker Inc., pg. 557-663, 1997).

Another object of the present invention are isolated nucleic acids encoding for the polypeptides of the invention having fibrillin-like activity, the polypeptides binding to an antibody or a binding protein generated against them, the corresponding fusion proteins, or mutants having antagonistic activity as disclosed above. Preferably, these nucleic acids should comprise a DNA sequence selected from the group consisting of SEQ ID NO: 1, SEQ ID NO: 5, SEQ ID NO: 12, the coding sequence of SEQ ID NO: 1 (the coding region starts at nucleotide 205 and ends at 1425), of SEQ ID NO: 5 (the coding region starts at nucleotide 205 and ends at 1590), of SEQ ID NO: 12 (the coding region starts at nucleotide 205 and ends at 1503), or the complement of said DNA sequences.

Alternatively, the nucleic acids of the invention should hybridize under high stringency conditions, or exhibit at least about 85% identity over a stretch of at least about 30 nucleotides, with a nucleic acid consisting of SEQ ID NO: 1, SEQ ID NO: 5, SEQ ID NO: 12, or be a complement of said DNA sequences.

The wording “high stringency conditions” refers to conditions in a hybridization reaction that facilitate the association of very similar molecules and consist in the overnight incubation at 60-65° C. in a solution comprising 50% formamide, 5×SSC (150 m M NaCl, 15 m M trisodium citrate), 50 mM sodium phosphate (p H 7 6), 5× Denhardt's solution, 10% dextran sulphate, and 20 microgram/ml denatured, sheared salmon sperm DNA, followed by washing the filters in O.1×SSC at the same temperature. These nucleic acids, including nucleotide sequences substantially the same, can be comprised in plasmids, vectors and any other DNA construct which can be used for maintaining, modifying, introducing, or expressing the encoding polypeptide. In particular, vectors wherein said nucleic acid molecule is operatively linked to expression control sequences can allow expression in prokaryotic or eukaryotic host cells of the encoded polypeptide.

The wording “nucleotide sequences substantially the same” includes all other nucleic acid sequences which, by virtue of the degeneracy of the genetic code, also code for the given amino acid sequences. In this sense, the literature provides indications on preferred or optimized codons for recombinant expression (Kane J F et al., 1995).

The nucleic acids and the vectors can be introduced into cells with different purposes, generating transgenic cells and organisms. A process for producing cells capable of expressing a polypeptide of the invention comprises genetically engineering cells with such vectors and nucleic acids.

In particular, host cells (e.g. bacterial cells) can be modified by transformation for allowing the transient or stable expression of the polypeptides encoded by the nucleic acids and the vectors of the invention. Alternatively, said molecules can be used to generate transgenic animal cells or non-human animals (by non-/homologous recombination or by any other method allowing their stable integration and maintenance), having enhanced or reduced expression levels of the polypeptides of the invention, when the level is compared with the normal expression levels. Such precise modifications can be obtained by making use of the nucleic acids of the inventions and of technologies associated, for example, to gene therapy (Meth. Enzymol., vol. 346, 2002) or to site-specific recombinases (Kolb A F, 2002). Model systems based on the fibrillin-like polypeptides disclosed in the present patent application for the systematic study of their function can be also generated by gene targeting into human cell lines (Bunz F, 2002).

Gene silencing approaches may also be undertaken to down-regulate endogenous expression of a gene encoding a polypeptide of the invention. RNA interference (RNAi) (Elbashir, S M et al, Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The polypeptides of the invention can be prepared by any method known in the art, including recombinant DNA-related technologies, and chemical synthesis technologies. In particular, a method for making a polypeptide of the invention may comprise culturing a host or transgenic cell as described above under conditions in which the nucleic acid or vector is expressed, and recovering the polypeptide encoded by said nucleic acid or vector from the culture. For example, when the vector expresses the polypeptide as a fusion protein with an extracellular or signal-peptide containing proteins, the recombinant product can be secreted in the extracellular space, and can be more easily collected and purified from cultured cells in view of further processing or, alternatively, the cells can be directly used or administered.

The DNA sequence coding for the proteins of the invention can be inserted and ligated into a suitable episomal or non-/homologously integrating vectors, which can be introduced in the appropriate host cells by any suitable means (transformation, transfection, conjugation, protoplast fusion, electroporation, calcium phosphate-precipitation, direct microinjection, etc.). Factors of importance in selecting a particular plasmid or viral vector include: the ease with which recipient cells that contain the vector, may be recognized and selected from those recipient cells which do not contain the vector, the number of copies of the vector which are desired in a particular host; and whether it is desirable to be able to “shuttle” the vector between host cells of different species.

The vectors should allow the expression of the isolated or fusion protein including the polypeptide of the invention in the Prokaryotic or Eukaryotic host cells under the control of transcriptional initiation/termination regulatory sequences, which are chosen to be constitutively active or inducible in said cell. A cell line substantially enriched in such cells can be then isolated to provide a stable cell line.

For Eukaryotic hosts (e.g. yeasts, insect, plant, or mammalian cells), different transcriptional and translational regulatory sequences may be employed, depending on the nature of the host. They may be derived form viral sources, such as adenovirus, bovine papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated. The cells stably transformed by the introduced DNA can be selected by introducing one or more markers allowing the selection of host cells which contain the expression vector. The marker may also provide for phototrophy to an auxotropic host, biocide resistance, e.g. antibiotics, or heavy metals such as copper, or the like. The selectable marker gene can either be directly linked to the DNA gene sequences to be expressed, or introduced into the same cell by co-transfection.

Host cells may be either prokaryotic or eukaryotic. Preferred are eukaryotic hosts, e.g. mammalian cells, such as human, monkey, mouse, and Chinese Hamster Ovary (CHO) cells, because they provide post-translational modifications to proteins, including correct folding and glycosylation. Also yeast cells can carry out post-translational peptide modifications including glycosylation. A number of recombinant DNA strategies exist which utilize strong promoter sequences and high copy number of plasmids which can be utilized for production of the desired proteins in yeast Yeast recognizes leader sequences in cloned mammalian gene products and secretes peptides bearing leader sequences (i.e., pre-peptides).

The above mentioned embodiments of the invention can be achieved by combining the disclosure provided by the present patent application on the sequence of novel fibrillin-like polypeptides with the knowledge of common molecular biology techniques.

Many books and reviews provides teachings on how to clone and produce recombinant proteins using vectors and Prokaryotic or Eukaryotic host cells, such as some titles in the series “A Practical Approach” published by Oxford University Press (“DNA Cloning 2: Expression Systems”, 1995; “DNA Cloning 4: Mammalian Systems”, 1996; “Protein Expression”, 1999; “Protein Purification Techniques”, 2001).

Moreover, updated and more focused literature provides an overview of the technologies for expressing polypeptides in a high-throughput manner (Chambers S P, 2002; Coleman T A, et al., 1997), of the cell systems and the processes used industrially for the large-scale production of recombinant proteins having therapeutic applications (Andersen D C and Krummen L, 2002, Chu L and Robinson D K, 2001), and of alternative eukaryotic expression systems for expressing the polypeptide of interest, which may have considerable potential for the economic production of the desired protein, such the ones based on transgenic plants (Giddings G, 2001) or the yeast Pichia pastoris (Lin Cereghino G P et al., 2002). Recombinant protein products can be rapidly monitored with various analytical technologies during purification to verify the amount and the quantity of the expressed polypeptides (Baker K N et al., 2002), as well as to check if there is problem of bioequivalence and immunogenicity (Schellekens H, 2002; Gendel S M, 2002).

Totally synthetic fibrillin-like polypeptides are disclosed in the literature and many examples of chemical synthesis technologies, which can be effectively applied for the fibrillin-like polypeptides of the invention given their short length, are available in the literature, as solid phase or liquid phase synthesis technologies. for example, the amino acid corresponding to the carboxy-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the carboxy-terminus to the amino-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), Cl-Z (2-chlorobenzyloxycarbonyl), Br-Z (2-bromobenzyloxycarbonyl), Bzl (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and C12-Bzl (2,6-dichlorobenzyl) for the amino groups; NO2 (nitro) and Pmc (2,2,5,7,8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

The purification of the polypeptides of the invention can be carried out by any one of the methods known for this purpose, i.e. any conventional procedure involving extraction, precipitation, chromatography, electrophoresis, or the like. A further purification procedure that may be used in preference for purifying the protein of the invention is affinity chromatography using monoclonal antibodies or affinity groups, which bind the target protein and which are produced and immobilized on a gel matrix contained within a column. Impure preparations containing the proteins are passed through the column. The protein will be bound to the column by heparin or by the specific antibody while the impurities will pass through. After washing, the protein is eluted from the gel by a change in pH or ionic strength. Alternatively, HPLC (High Performance Liquid Chromatography) can be used. The elution can be carried using a water-acetonitrile-based solvent commonly employed for protein purification.

The disclosure of the novel polypeptides of the invention, and the reagents disclosed in connection to them (antibodies, nucleic acids, cells) allows also to screen and characterize compounds that enhance or reduce their expression level into a cell or in an animal.

“Oligonucleotides” refers to either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

The invention includes purified preparations of the compounds of the invention (polypeptides, nucleic acids, cells, etc.). Purified preparations, as used herein, refers to the preparations which contain at least 1%, preferably at least 5%, by dry weight of the compounds of the invention.

The present patent application discloses a series of novel fibrillin-like polypeptides and of related reagents having several possible applications. In particular, whenever an increase in the fibrillin-like activity of a polypeptide of the invention is desirable in the therapy or in the prevention of a disease, reagents such as the disclosed fibrillin-like polypeptides, the corresponding fusion proteins and peptide mimetics, the encoding nucleic acids, the expressing cells, or the compounds enhancing their expression can be used.

Therefore, the present invention discloses pharmaceutical compositions for the treatment or prevention of diseases needing an increase in the fibrillin-like activity of a polypeptide of the invention, which contain one of the disclosed fibrillin-like polypeptides, the corresponding fusion proteins and peptide mimetics, the encoding nucleic acids, the expressing cells, or the compounds enhancing their expression, as active ingredient. The process for the preparation of these pharmaceutical compositions comprises combining the disclosed fibrillin-like polypeptides, the corresponding fusion proteins and peptide mimetics, the encoding nucleic acids, the expressing cells, or the compounds enhancing their expression, together with a pharmaceutically acceptable carrier. Methods for the treatment or prevention of diseases needing an increase in the fibrillin-like activity of a polypeptide of the invention, comprise the administration of a therapeutically effective amount of the disclosed fibrillin-like polypeptides, the corresponding fusion proteins and peptide mimetics, the encoding nucleic acids, the expressing cells, or the compounds enhancing their expression.

Amongst the reagents disclosed in the present patent application, the ligands, the antagonists or the compounds reducing the expression or the activity of polypeptides of the invention have several applications, and in particular they can be used in the therapy or in the diagnosis of a disease associated to the excessive fibrillin-like activity of a polypeptide of the invention.

Therefore, the present invention discloses pharmaceutical compositions for the treatment or prevention of diseases associated to the excessive fibrillin-like activity of a polypeptide of the invention, which contain one of the ligands, antagonists, or compounds reducing the expression or the activity of such polypeptides, as active ingredient The process for the preparation of these pharmaceutical compositions comprises combining the ligand, the antagonist, or the compound, together with a pharmaceutically acceptable carrier. Methods for the treatment or prevention of diseases associated to the excessive fibrillin-like activity of the polypeptide of the invention, comprise the administration of a therapeutically effective amount of the antagonist, the ligand or of the compound.

The pharmaceutical compositions of the invention may contain, in addition to fibrillin-like polypeptide or to the related reagent, suitable pharmaceutically acceptable carriers, biologically compatible vehicles and additives which are suitable for administration to an animal (for example, physiological saline) and eventually comprising auxiliaries (like excipients, stabilizers, adjuvants, or diluents) which facilitate the processing of the active compound into preparations which can be used pharmaceutically.

The pharmaceutical compositions may be formulated in any acceptable way to meet the needs of the mode of administration. For example, of biomaterials, sugar-macromolecule conjugates, hydrogels, polyethylene glycol and other natural or synthetic polymers can be used for improving the active ingredients in terms of drug delivery efficacy. Technologies and models to validate a specific mode of administration are disclosed in literature (Davis B G and Robinson M A, 2002; Gupta P et al., 2002; Luo B and Prestwich G D, 2001; Cleland J L et al., 2001; Pillai O and Panchagnula R, 2001).

Polymers suitable for these purposes are biocompatible, namely, they are non-toxic to biological systems, and many such polymers are known. Such polymers may be hydrophobic or hydrophilic in nature, biodegradable, non-biodegradable, or a combination thereof. These polymers include natural polymers (such as collagen, gelatin, cellulose, hyaluronic acid), as well as synthetic polymers (such as polyesters, polyorthoesters, polyanhydrides). Examples of hydrophobic non-degradable polymers include polydimethyl siloxanes, polyurethanes, polytetrafluoroethylenes, polyethylenes, polyvinyl chlorides, and polymethyl methaerylates. Examples of hydrophilic non-degradable polymers include poly(2-hydroxyethyl methacrylate), polyvinyl alcohol, poly(N-vinyl pyrrolidone), polyalkylenes, polyacrylamide, and copolymers thereof. Preferred polymers comprise as a sequential repeat unit ethylene oxide, such as polyethylene glycol (PEG).

Any accepted mode of administration can be used and determined by those skilled in the art to establish the desired blood levels of the active ingredients. For example, administration may be by various parenteral routes such as subcutaneous, intravenous, intradermal, intramuscular, intraperitoneal, intranasal, transdermal, oral, or buccal routes. The pharmaceutical compositions of the present invention can also be administered in sustained or controlled release dosage forms, including depot injections, osmotic pumps, and the like, for the prolonged administration of the polypeptide at a predetermined rate, preferably in unit dosage forms suitable for single administration of precise dosages.

Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyl oleate or triglycerides. Aqueous injection suspensions that may contain substances increasing the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99.99 percent, preferably from about 20 to 75 percent of active compound together with the excipient.

The wording “therapeutically effective amount” refers to an amount of the active ingredients that is sufficient to affect the course and the severity of the disease, leading to the reduction or remission of such pathology. The effective amount will depend on the route of administration and the condition of the patient.

The wording “pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution. Carriers can be selected also from starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the various oils, including those of petroleum, animal, vegetable or synthetic origin (peanut oil, soybean oil, mineral oil, sesame oil).

It is understood that the dosage administered will be dependent upon the age, sex, health, and weight of the recipient, kind of concurrent treatment, if any, frequency of treatment, and the nature of the effect desired. The dosage will be tailored to the individual subject, as is understood and determinable by one of skill in the art. The total dose required for each treatment may be administered by multiple doses or in a single dose. The pharmaceutical composition of the present invention may be administered alone or in conjunction with other therapeutics directed to the condition, or directed to other symptoms of the condition. Usually a daily dosage of active ingredient is comprised between 0.01 to 100 milligrams per kilogram of body weight per day. Ordinarily 1 to 40 milligrams per kilogram per day given in divided doses or in sustained release form is effective to obtain the desired results. Second or subsequent administrations can be performed at a dosage, which is the same, less than, or greater than the initial or previous dose administered to the individual.

Apart from methods having a therapeutic or a production purpose, several other methods can make use of the fibrillin-like polypeptides and of the related reagents disclosed in the present patent application.

In a first example, a method is provided for screening candidate compounds effective to treat a disease related to a fibrillin-like polypeptide of the invention, said method comprising:
(a)contacting host cells expressing such polypeptide, transgenic non-human animals, or transgenic animal cells having enhanced or reduced expression levels of the polypeptide, with a candidate compound and
(b)determining the effect of the compound on the animal or on the cell.

In a second example there is provided a method for identifying a candidate compound as an antagonist/inhibitor or agonist/activator of a polypeptide of the invention, the method comprising:
(a) contacting the polypeptide, the compound, and a mammalian cell or a mammalian cell membrane; and
(b) measuring whether the molecule blocks or enhances the interaction of the polypeptide, or the response that results from such interaction, with the mammalian cell or the mammalian cell membrane.

In a third example, a method for determining the activity and/or the presence of the polypeptide of the invention in a sample, can detect either the polypeptide or the encoding RNA/DNA. Thus, such a method comprises:
(a) providing a protein-containing sample;
(b) contacting said sample with a ligand of the invention; and
(c) determining the presence of said ligand bound to said polypeptide, thereby determining the activity and/or the presence of polypeptide in said sample.

In an alternative, the method comprises:
(a) providing a nucleic acids-containing sample;
(b) contacting said sample with a nucleic acid of the invention; and
(c) determining the hybridization of said nucleic acid with a nucleic acid into the sample, thereby determining the presence of the nucleic acid in the sample.

In this sense, a primer sequence derived from the nucleotide sequences presented in SEQ ID NO:1, SEQ ID NO: 5, or SEQ ID NO: 12, can be used as well for determining the presence or the amount of a transcript or of a nucleic acid encoding a polypeptide of invention in a sample by means of Polymerase Chain Reaction amplification.

A further object of the present invention are kits for measuring the activity and/or the presence of fibrillin-like polypeptide of the invention in a sample comprising one or more of the reagents disclosed in the present patent application: a fibrillin-like polypeptide of the invention, an antagonist, ligand or peptide mimetic, an isolated nucleic acid or the vector, a pharmaceutical composition, an expressing cell, or a compound increasing or decreasing the expression levels.

Such kits can be used for in vitro diagnostic or screenings methods, and their actual composition should be adapted to the specific format of the sample (e.g. biological sample tissue from a patient), and the molecular species to be measured. For example, if it is desired to measure the concentration of the fibrillin-like polypeptide, the kit may contain an antibody and the corresponding protein in a purified form to compare the signal obtained in Western blot. Alternatively, if it is desired to measure the concentration of the transcript for the fibrillin-like polypeptide, the kit may contain a specific nucleic acid probe designed on the corresponding ORF sequence, or may be in the form of nucleic acid array containing such probe. The kits can be also in the form of protein-, peptide mimetic-, or cell-based microarrays (Templin M F et al., 2002; Pellois J P et al., 2002; Blagoev B and Pandey A, 2001), allowing high-throughput proteomics studies, by making use of the proteins, peptide mimetics and cells disclosed in the present patent application.

The present patent application discloses novel fibrillin-like polypeptides and a series of related reagents that may be useful, as active ingredients in pharmaceutical compositions appropriately formulated, in the treatment or prevention of diseases and conditions such as those in which skin is damaged from aging, injuries or the sun, or for restoring skin damaged from the same, also multiple sclerosis, cancer, bone, joint or ligament reconstruction after fractures or lesions, osteoarthritis, rheumatoid arthritis, osteoporosis, cardiovascular diseases and fibrosis (including liver fibrosis, kidney fibrosis, hepatitis and renal disorders).

The therapeutic applications of the polypeptides of the invention and of the related reagents can be evaluated (in terms or safety, pharmacokinetics and efficacy) by the means of the in vivo/in vitro assays making use of animal cell, tissues and or by the means of in silico/computational approaches (Johnson D E and Wolfgang G H, 2000), known for the validation of fibrillin-like polypeptides and other biological products during drug discovery and preclinical development

Therapeutic Uses

SCS0008 nucleic acid molecules, polypeptides, and agonists and antagonists thereof can be used to treat, diagnose, ameliorate, or prevent a number of diseases, disorders, or conditions, including those recited herein.

SCS0008 polypeptides' agonists and antagonists include those molecules which regulate SCS008 polypeptides' activity and either increase or decrease at least one activity of the mature form of the SCS0008 polypeptides. Agonists or antagonists may be co-factors, such as a protein, peptide, carbohydrate, lipid, or small molecular weight molecule, which interact with the SCS0008 polypeptides and thereby regulate their activity.

Potential polypeptide agonists or antagonists include antibodies that react with either soluble or membrane-bound forms of the SCS0008 polypeptides. Molecules that regulate SCS0008 polypeptides' expression typically include nucleic acids encoding the SCS0008 polypeptides that can act as anti-sense regulators of expression.

Morimura et al. and Brandenberger et al. showed that nephronectin (or POEM) is a novel ligand for α₈β₁ integrin. As SCS0008 seems to be the human ortholog of nephronectin and as the SCS0008 polypeptides do contain a RGD integrin binding tripeptide (example 4), SCS0008 is likely to act as well as a ligand for α₈β₁ integrin. Since it is known that α₈β₁ integrin is implicated in the development of particular diseases, SCS0008, as a ligand, might as well act as a contributing factor in the onset of those disorders. In addition, Morimura et al. And Brandenberger et al. have showed that nephronectin is expressed in many tissues, including the organs and tissues mentioned below. As such, the present SCS0008 polypeptides are not only useful in the tissues and organs mentioned hereafter, but also in all of those mentioned by Morimura et al. and Brandenberger et al.

Morimura et al. and Brandenberger et al. indicate that nephronectin is involved in kidney morphogenesis and function. Miner Jeffrey suggests that nephronectin may play a role in establishing or maintaining the filtration barrier (Jeffrey H. Miner, The Journal of Cell Biology, Volume 154, Number 2, Jul. 23, 2001, pp. 257-259). Miner Jeffrey further points out that nephronectin and laminin-10 appear to be colocalized in the Wolffian duct and ureteric bud basement membranes, it is possible that they may interact with each other and/or cooperate to promote efficient ureteric bud outgrowth and branching, either trough parallel or common pathways. In addition, Brandenberger et al. points out that secreted Glial cell-derived neurotrophic factor (GDNF) has been shown to control in-growth of the ureteric bud and that α₈β₁ is likely involved in this signalling pathway. It is also known that GDNF-ligand GFRα1 (also GFRα2, GFRα3, GFRα4) activates RET tyrosine kinase. Schuchardt et al. found that RET is required for ureteric bud growth and branching. Eya1 has also been involved in human congenital anomalies of the kidney and urinary tract (CAKUT; Nakanishi K, Yoshikawa N. Pediatr Int. October 2003; 45(5):610-6.). As such SCS0008 nucleic acid molecules, polypeptides, and agonists and antagonists thereof and particularly SCS0008-SV2 (which is a splice variant from the kidney, example 3) may be useful in diagnosing or treating renal branching defects, cysts, stone formation, vesicouretal reflux, renal failure, defects in the filtration barrier, hydronephrosis, congenital anomalies of the kidney and urinary tract (CAKUT) such as bilateral renal agenesis. SCS0008 antagonists and particularly SCS0008-SV2 antagonists (e.g. antibodies targeted to SCS0008) may be useful in diagnosing or treating kidney cancers, particularly renal dysplasia or hypoplasia

Lu et al. (Lu M et al., J Cell Sci. Dec. 1, 2002; 115(Pt 23) :4641-8) showed that α₈β₁ integrin is expressed by alveolar interstitial cells in normal lung and is upregulated during the development of fibrosis. Binding of another ligand of α₈β₁ integrin, Latency-Associated Peptide (LAP)-Transforming Growth Factor-beta 1 (TGFbeta1), results in cell proliferation and might therefore be important in the development of fibrosis. Without whishing to be bound to theory, SCS0008, likewise, by α₈β₁ integrin binding, might be involved in this cell proliferation. In addition, Sparrow and Lamb state that GDNF appears to be produced by airway smooth muscle (ASM), which is an integral component of the primordial lung, and might be involved in smooth muscle myogenesis (Sparrow M P, Lam J P. Respir Physiol Neurobiol. Sep. 16, 2003; 137(2-3):361-72). Levine et al. demonstrated de novo expression of α₈β₁ integrin in activated hepatic stellate cells, the myofibroblasts equivalent in liver (Levine D et al., Am J Pathol. June 2000; 156(6):1927-35). Bauhau et al. suggest a role of GDNF in neurofibromatosis type 1 (see Online Mendelian Inheritance in Man (OMIM), http://www.ncbi.nlm.nih.gov/entrez/guery.fcgi?db=OMIM, OMIM*600837). Neurofibromatosis shows a diverse variety of clinical synopsis including Macrocephaly, Sphenoid dysplasia, Lisch nodules (iris hamartomas), Glaucoma, Hypertelorism, Renal artery stenosis, Hypertension, Scoliosis, Spina bifida, Pseudoarthrosis, Thinning of long bone cortex, Local bony overgrowth, Absent patellae, Neurofibromas, Plexiform neurofibroma, Cafe-au-lait spots, Axillary freckling, Inguinal freckling, Mental retardation, 30% learning disabilities, 10% mild mental retardation, Aqueductal stenosis, Hydrocephalus, neoplasia including Optic glioma, Meningioma, Hypothalamic tumor, Neurofibrosarcoma, Rhabdomyosarcoma, Duodenal carcinoid, Somatostatinoma, Parathyroid adenoma, Pheochromocytoma, Tumors at multiple other sites including CNS. As such, the SCS0008 polypeptides of the invention, agonists or antagonists thereof may also be useful in reducing or in the diagnosis or treatment of the individual or combined symptoms or clinical outcome of neurofibromatosis. In addition, SCS0008 nucleic acid molecules, polypeptides, and agonists and antagonists thereof and particularly SCS0008-SV1 (which is a splice variant from the lung, example 3) may be useful in diagnosing or treating fibrosis, preferably pulmonary fibrosis, liver fibrosis, neurofibromatosis type 1, Watson syndrome, or persistent acute respiratory distress syndrome (ARDS). In addition, SCS0008 nucleic acid molecules, polypeptides, and agonists and antagonists thereof and particularly SCS0008-SV1 may be useful in smooth muscle myogenesis.

Brandenberger et al. suggests that nephronectin acting through α₈β₁ could induce a signalling pathway that results in enhanced expression of GDNF or, alternatively, it could synergize with GDNF by promoting secretion of this factor or its localization in the extracellular matrix. Since it is known that GDNF is implicated in the development of particular diseases, SCS0008, as a potential ligand or at least by enhancing GDNF's expression, might as well act as a contributing factor in the onset of those disorders. Matti Airaksinen and Mart Saarma have reviewed the role of the GDNF family in the brain (Matti S. Airaksinen and Mart Saarma, Nature Reviews, Volume 3, May 2002, pp. 383-394). They point out that GDNF is implicated in motor neuron disease or motor neuron injury (GDNF can support long-term motor neuron survival and axon regeneration after peripheral nerve injury), in sensory regeneration (GDNF produces potent analgesic effect in neuropathic models of pain and can cause sensory axons to regenerate back into the spinal chord; local GDNFexpression induces Schwann cell migration to the lesion site, leading to remyelination of regenerating axons after injury, Blesch A et al. J Comp Neurol. Dec. 15, 2003; 467(3):403-17), sensory disorders including diabetic neuropathy, neuropathic pain, in ischaemia or stroke (GDNF administered before or just after anoxia can reduce ischaemic brain injury), preferably GDNF should be administered in the early phase of stroke, in epilepsy (GDNF might modulate seizure susceptibility), in neurodegenerative disorders such as Parkinson's disease or multiple sclerosis (GDNF can prevent the neuro toxin-induced death of dopamine neurons and can promote functional recovery; preferably GDNF is co-administered with heparin; see also Jeffrey H. Kordower, Ann Neurol 2003, 53 (suppl 3):S120-134, and Blesch et al.), and finally in addiction (infusion of GDNF into the VTA blocks certain biochemical adaptations to chronic cocaine and morphine, as well as the rewarding effects of cocaine). Jollivet et al. show that GDNF-releasing microspheres could be a promising strategy in the treatment of Parkinson's disease (Jollivet C et al. Biomaterials. February 2004; 25(5):933-42). In a similar way, SCS0008-releasing microspheres could be used. Nagano et al. suggest that GDNF could be used for sciatic nerve ligation (chronic constrictive injury) or spinal nerve ligation (Nagano N et al. Br J Pharmacol. December 2003; 140(7):1252-60). Tai et al. suggest that GDNF could be used to treat spinal cord injury (SCI) or spinal cord contusion (Tai M H et al. Exp Neurol. October 2003; 183(2):508-15). Ochiai H et al. state that local administration of GDNF in a neonatal preganglionic Erb's palsy model result in significant improvement in deficits (Ochiai H et al. Neurosurgery. October 2003; 53(4):973 -7). Aszman et al. state that exogenous trophic support of motoneurons (e.g. GNDF or/and BDNF) might have a role in the treatment of all types of severe neonatal plexopathies (e.g. obstetric brachial plexus lesions; Aszmann O C et al. Plast Reconstr Surg. Sep. 15, 2002; 110(4):1066-72). Tolbert and Clark indicate that GDNF can delay the onset of hereditary Purkinje cell degeneration and gait ataxia (Tolbert D L, Clark B R. Exp Neurol. September 2003; 183(1):205-19). Schwann cells are predisposed to develop schwannoma (Bartolami S et al. J Neurobiol. December 2003; 57(3):270-90). As such, antagonists (antibodies) of GDNF or SCS0008 could be used to treat schwannoma or, by extension, other brain-related cancers. Perez-Garcia et al. state that changes of [Ca2+]1, induced by GDNF, promote neuronal survival through a mechanism that involves a direct regulation of PI 3-kinase activation by calmodulin thus suggesting a central role for Ca2+ and calmodulin in the signalling cascade for neuronal survival mediated by neurotrophic factors (Perez-Garcia M J et al. J Biol Chem. Nov. 20, 2003, Epub; see also Wang J et al. Neurosignals. March-April 2003; 12(2):78-88). As SCS0008 polypeptides contain three Calcium-binding EGF-like domains, the involvement of SCS0008 polypeptides in the above mechanism and thus in neuronal survival is strengthened. McBride et al. show that the viral-mediated gene transfer of GDNF into the striatum provides neuroanatomical and behavioural protection in a rodent model of Huntington's disease (McBride J L et al. Exp Neurol. June 2003; 181(2):213-23). Alberch et al. suggest that neurotrophic factors (e.g. GDNF) may be able to provide selective protection for basal ganglia circuits, which are affected in striatonigral degenerative disorders, such as Huntington's disease or multisystem atrophy (Alberch J et al. Brain Res Bull. April 2002; 57(6):817-22). Laminin-10 and β1 integrins have also been involved in neuronal survival (Chen Zu-Lin et al. Molecular Biology of the Cell. July 2003 Vol. 14:2665-2676). Chen et al. state that laminin-10 protects against neuronal death. As such SCS0008 nucleic acid molecules, polypeptides, and agonists and antagonists thereof may be useful in diagnosing or treating degenerative disorders, striatonigral degenerative disorders, Huntington's disease, multisystem atrophy.

As such SCS0008 nucleic acid molecules, polypeptides, and agonists and antagonists thereof may be useful in the diagnosing or treating motor neuron diseases, motor neuron injuries, in favorising sensory regeneration, in sensory disorders including diabetic neuropathy, neuropathic pain, in ischaemia or stroke (preferably SCS0008 should be administered in the early phase of stroke), in epilepsy, in neurodegenerative disorders such as Parkinson's disease or multiple sclerosis (preferably SCS0008 is co-administered with heparin and/or by the means of microspheres), in striatonigral degenerative disorders, Huntington's disease, multisystem atrophy, in sciatic nerve ligation (chronic constrictive injury), spinal nerve ligation, spinal cord injury (SCI) or spinal cord contusion, in neonatal preganglionic Erb's palsy, in neonatal plexopathies such as obstetric brachial plexus lesions; in hereditary Purkinje cell degeneration and gait ataxia. SCS0008 could be administered alone or in combination with neurotrophic factors (e.g. neurturin, artemin, persephin, nerve growth factor, brain-derived neurotrophic factor, GDNF), heparin or other therapeutic agent Antagonists (e.g. antibodies) of SCS0008 could be used to treat schwannoma or other brain-related cancers.

GDNF seems to be involved in Hirschsprung disease (HSCR), which is a congenital disorder characterized by an absence of ganglion cells in the nerve plexus of the lower digestive tract (Garcia-Barcelo M et al. Clin Chem. Nov. 18, 2003. Epub; see also Benailly H K et al. Clim Genet September 2003; 64(3):204-9; see also OMIM*600837). As such SCS0008 nucleic acid molecules, polypeptides, and agonists and antagonists thereof may be useful in diagnosing or treating Hirschsprung disease (HSCR), central hypoventilation syndrome (ondine curse).

Signal from the epidermal growth factor (EGF) receptor family are thought to combine with integrin-dependent adhesion to laminins to contribute to disease progression and metastasis in cancer (Zamurs I et al. Biomed Chromatogr. March-April 2003; 17(2-3):201-1 1). Zamurs et al. showed that a colon cancer cell line secretes and adheres to laminin-10 through multiple integrin receptors, and that EGF stimulates spreading and migration of these cells on the same substrate. Laminin-10/11 was also shown to be present at the invasive edge of moderately differentiated colon cancers. Gu et al report that laminin 10-11 strongly promotes migration of human lung carcinoma cells by activating a β1 integrin pathway (Gu J et al. Exp Dermatol. October 2002; 11 (5):387-97). Laminin and integrins have also been implicated in pancreas function. Funahashi et al. showed that expression of some of the integrin subunits in pancreatic cancer cells was enhanced by GDNF. This enhancement and associate increase in adhesive and invasive ability by GDNF were inhibited by blocking the GDNF receptor or the integrin betal subunit (Funahashi H et al. Pancreas. August 2003; 27(2):190-6). Okada et al. report also that GDNF promotes pancreatic cancer cell invasion in vitro (Okada Y et al. Surgery. August 2003; 134(2):293 -9). Japon et al. (2002) suggest that GDNF could be involved in pituitary tumors, more specifically adenomas, corticotropinomas, somatotropinomas, prolactinomas (see OMIM*600837). The RET and eventually GDNF genes have been associated with multiple endocrine neoplasia, type IIA and type IIB as well as medullary thyroid carcinoma (OMIM*164761 and OMIM*162300). Similar to nephronectin, Eyes absent 1 (EYA1) homozygosity result in an absence of ureteric bud outgrowth and a subsequent failure of metanephric induction (OMIM*601653). In addition, GDNF expression was not detected in Eya1 −/− metanephric mesenchyme (OMIM*601653). As such, nephronectin and Eya1 might interact Eya1 is involved in branchiootorenal dysplasia and branchiootic syndrome. As such SCS0008 antagonists (e.g. antibodies targeted to SCS0008) as well as SCS0008-SV1 and SCS008-SV2 antagonists (which are preferabily used for lung or kidney cancers respectively) may be useful in diagnosing or treating pancreatic cancer, pituitary tumors, more specifically adenomas, corticotropinomas, somatotropinomas, prolactinomas, and multiple endocrine neoplasia, type IIA and type IIB, medullary thyroid carcinoma, lung carcinoma, papillary thyroid carcinoma, colonic aganglionosis, colon cancer, MEN2-associated tumors, pheocromocytoma, amyotrophic lateral sclerosis, branchiootorenal dysplasia and branchiootic syndrome.

In view of the possible interaction or cooperation between laminin-10 and nephronectin (see above), disorders were laminin-10 is involved may be treated or diagnosed with the SCS0008 polypeptides of the present invention. Spessotto et al. showed that the preferential locomotion of leukemic cells was directed towards laminin isoform 8 and 10 (Spessotto P et al. Matrix Biol. June 2003; 22(4):351-61). The motility-promoting interaction with these laminins was mediated by β1 integrins. In addition, Yu and Talts point out that both integrins and α-dystroglycan are: expressed on cancer cells, and binding to laminin-10/11 may therefore be important for cell movement through endothelial and epithelial basement membranes and thus for metastasis (Yu H and Tatis J F. Biochem J. 2003. 371:289-299). They also add that Ca2+ ion appear to be in some way involved in the interaction between α-dystroglycan and laminin10/11 through α5LG4-5. Without wishing to be bound to theory, nephronectin could have a role in this interaction via its calcium-binding EGF domains. Li et al. showed that laminin-10 can restore hair follicle development, thus enabling correction of cutaneous developmental defects (Li J et al. Embo J. May 15, 2003 ; 22(10):2400 -10). Laminins and β1 integrins have also been implicated in hematopoiesis (Gu Y C et al. Blood. Feb. 1, 2003 ; 101(3):877-85) as well as in angiogenesis in wound repair (Li J et al. Microsc Res Tech. Jan. 1, 2003 ; 60(1):107-14). Kikkawa et al. point out that the Lutheran blood group glycoprotein (Lu) is an Ig superfamily transmembrane receptor for lamin α5 and that Lu is thought to be involved in both normal and disease processes, including sickle cell disease and cancer (Kikkawa Y et al. J Biol Chem. Nov. 22, 2002; 277(47):44864-9). They showed that Lu binds specifically to laminin 10/11 in vivo and in vitro. In addition, Laminin-beta1 has been implicated in neonatal cutis laxa, marfanoid or marfan syndrome and arachnodactyly (OMIM*150240).

As such SCS0008 antagonists (e.g. antibodies targeted to SCS0008) as well as SCS0008-SV1 and SCS008-SV2 antagonists (which are preferabily used for lung or kidney cancers respectively) may be useful in diagnosing or treating leukaemia, in the reduction or inhibition of metastasis in cancer.

As such SCS0008 nucleic add molecules, polypeptides, and agonists and antagonists thereof may be useful in diagnosing or treating cutaneous developmental defects, in hematopoiesis-related diseases, in sickle cell disease, in neonatal cutis laxa, marfanoid or marfan syndrome and arachnodactyly and in favourising angiogenesis in wound repair.

Yomogida et al. showed a dramatic amplification of germinal stem cells (GSC) in mouse testis following transfection of human GDNF cDNA into Sertoli cells (Yomogida K et al. Biol Reprod. October 2003; 69(4):1303-7). As such SCS0008 nucleic acid molecules, polypeptides, and agonists and antagonists thereof may be useful in the amplification of germinal stem cells.

Sariola and Meng state that GDNF is expressed by Sertoli cells and showed that GDNF regulates the cell fate decision of undifferentiated spermatogonia. In addition, they show that in mice overexpressing GDNF in testes, undifferentiated spermatogonia accumulate in the tubules, no sperm is produced, and the mice are infertile. After a year, they observe that GDNF overexpressing mice frequently (89%) develop testicular tumors, amd most of them are bilateral (56%), the tumors mimicking classical seminomas in men. As such SCS0008 antagonists (e.g. antibodies targeted to SCS0008) may be useful in the diagnosis or treatment of male infertility, seminomas. SCS0008 nucleic acid molecules, polypeptides, and agonists and antagonists thereof may be useful in the development, proliferation, and in the differentiation of spermatogonia.

Thibault et al. (Thibault G et al., Am J Physiol Cell Physiol. November 2001; 281(5):C1457-67) observed that rat cardiac fibroblasts harbor α₈β₁ integrin. In addition, stimulation of cardiac fibroblasts by angiotensin II (ANG II) or TGF β1 resulted in an increase of protein and heightening by 50% of the receptor density α₈β₁ integrin. Low nephron number, inherited or acquired, has been linked to increased risk, not only of renal failure, but also of hypertension (Cullen-McEwen L A et al. Hypertension. February 2003; 41(2):335-40). Cullen et al. show that GDNF heterozygous mice have elevated arterial pressure, glomerular hypertrophy and hyperfiltration. Wintour et al. point out that GDNF is a factor that predispose to the onset of adult hypertension and of cardiovascular disease (Wintour E M et al. Placenta. April 2003; 24 Suppl A:S65-71). As such SCS0008 nucleic acid molecules, polypeptides, and agonists and antagonists thereof may be useful in diagnosing or treating hypertension or cardiovascular diseases.

Chen et al. show that increased levels of GDNF exclusively in the target rescue 30% of oculomotor neurons that would normally die during developmental cell death, a rate of rescue similar to that with systemic GDNF application (Chen J et al. Mol Cell Neurosci. September 2003; 24(1):41-56). Wordinger et al. state that a potential for autocrine and/or paracrine GDNF signalling exists within the lamina cribrosa, a tissue involved in glaucoma pathogenesis (Wordinger R J et al. Mol Vis. Jun. 16, 2003; 9:249-56). In addition, Rothermel et Layer suggest that GDNF, at least in vitro, affects rod receptors (Rothermel A, Layer P G. Invest Ophtalmol Vis Sci. May 2003; 44(5):2221 -8). Depending on the developmental stage, GDNF regulates their proliferation, differentiation, and survival. Karlsson et al. show that GDNF labelling is mainly found in chicken embryonic day 4-5 retina but weak labelling could also be found over scattered retinal cells at later cells (Karisson M et al. Mech Dev. June 2002; 114(1-2):161-5). They also show that c-ret labelling is found over ganglion cells, amacrine and horizontal cells; GRF alpha 1 over amacrine and horizontal cells; and GFR alpha 2 over ganglion cells, amacrine cells and photoreceptors. Ljubimov et al. showed that basement membrane (BM) components, especially fibronectin and lamini-10, in postcataract surgery (PCS) corneas were altered to a great extent (40-60%). In addition, they found that tenascin-C and fibrillin-1 were mostly found in diseased but not in PCS corneas (Ljubimov A V et al. Cornea. January 2002; 21(1):74-80). Eya1 has also been implicated in cataract (see OMIM*601653). As such SCS0008 nucleic acid molecules, polypeptides, and agonists and antagonists thereof may be useful in the development, proliferation, differentiation and survival of oculomotor neurons, photoreceptors and particularly rod receptors (rosetted spheroids), ganglion cells, amacrine and horizontal cells or useful in diagnosing or treating corneal edematous diseases, Fuch's dystrophy comeas, cataract or eye injuries. SCS0008 antagonists (e.g. antibodies targeted to SCS008) may be useful in diagnosing or treating glaucoma.

Gladson et al. established that α₈β₁ integrin is expressed on neonatal rat astrocytes. They demonstrate also that unstimulated primary neonatal rat astrocytes attach to vitronectin, utilizing α₈β₁ and α₅β₅ integrins, and that this attachment is regulated by the type 1 Plasminogen Activator Inhibitor (PAI-1). As such SCS0008 nucleic acid molecules, polypeptides, and agonists and antagonists thereof may be useful in diagnosing or treating germinal matrix hemorrhage and infarction.

Littlewood et al. suggest that α₈β₁ integrin regulates hair cell differentiation and stereocilia maturation and that mutations in integrin genes may lead to inner-ear diseases (Uttlewood Evans A et al., Nat Genet. April 2000; 24(4):424-8). Furthermore, GDNF is suggested to play a role in the postnatal inner ear (GDNF expression found in the cochlea) in addition to its role during development (Stankovic K M, Corfas G. Hear Res. November 2003; 185(1-2):97-108). Kawamoto et al. suggest that adenovirus-demiated overexpression of GDNF and TGF-betal can be used in combination to protect cochlear hair and hearing from ototoxic trauma (Kawamoto K et al. Mol Ther. April 2003; 7(4):484-92). They also point out that GDNF overexpression in the inner ear can protect hair cells against degeneration induced by ototoxicity. As such SCS0008 nudeic acid molecules, polypeptides, and agonists and antagonists thereof alone or in combination with TGF-beta1 may be useful in diagnosing or treating inner-ear diseases; inner-ear injuries (e.g. ototoxic trauma), scala tympani fibrosis, or for the development, differentiation, and survival of inner hair cells (cochlear hair cells).

Moursi et al. showed that direct osteoblast interactions with the extrace Iluar matrix are mediated by a selected group of integrin receptors that includes alpha5ss1, alpha3ss1, alpha8ss1. Furthemore, Morimura et al. suggest a role of POEM in the early stage of osteoblastic cell differentiation and that POEM may play important roles in osteoblastic function by sending survival signals via α₈β₁ integrin and mediating cell-cell interaction. POEM was expessed in the endocrine organs (parathyroid gland, thyroid gland, hypophysis, and pineal organ), which are closely related to growth, bone metabolism, and calcium and phosphorus homeostasis. They add that the data suggest a relationship between POEM and calcium metabolism. In addition, the SCS0008 contain three EGF-calcium binding motifs. As such SCS0008 nucleic acid molecules, polypeptides, and agonists and antagonists thereof may be useful in diagnosing or treating osteoporosis-pseudoglioma syndrome, osteopetrosis, endosteal hyperostosis, osteosclerosis, high bone mass disorder, dolichostenomelia, micrognathia, congenital kyphoscoliosis, retrognathiascoliosis, thoracic lordosis, sphondylolisthesis, lumbosacral dural ectasia, in growth defetcs, bone metabolism, and calcium and phosphorus homeostasis as well as osteoblastic cell differentiation.

Several domains have been identified in the SCS0008 protein, which are indicators of SCS0008's associated diseases. Using SMART (Simple Modular Architecture Research Tool, which allows the identification and annotation of genetically mobile domains and the analysis of domain architectures, http://smart.embl-heidelberg.del) and its OMIM (Online Mendelian Inheritance in Man, which is a database cataloging human genes and genetic disorders, http://www.ncbi.nim.nih.gov/entrez/query.fcgi?db=OMIM) curated human diseases associated with missense mutations within domains, it can be retrieved human diseases associated with domains found in SCS0008 (see example 4). Search for OMIM human diseases associated with the EGF-like or the calcium-binding EGF domains were performed. Some of the diseases have already been mentioned above and strengthens the likelihood that the SCS0008 polypeptides of the invention may be useful for the diagnosis or treatment of these diseases.

As such SCS0008 nucleic acid molecules, polypeptides, and agonists and antagonists (antagonists of SCS0008 as antibodies being useful for all types of cancer mentioned below) thereof may be useful in diagnosing or treating Severe classic or classic or mild variable or neonatal form or atypical Marfan syndrome, aortic aneurysm, marfanoid skeletal syndrome, Weill-Marchesani syndrome, MASP2 deficiency, Factor 1× deficiency, megaloblastc anaemia, pseudoachondroplasia, epiphyseal dysplasia, cutis laxa, lissencephaly syndrome (preferably Norman-Roberts type), osteoporosis-pseudoglioma syndrome, osteopetrosis, endosteal hyperostosis, osteosclerosis, high bone mass disorder, dolichostenomelia, micrognathia, congenital kyphoscoliosis, recurrent venous or mesenteric or cerebral venous or arterial thrombosis, superficial thrombophlebibs, Warfarin-induced skin necrosis, retrognathiascoliosis, thoracic lordosis, sphondylolisthesis, lumbosacral dural ectasia, Beals syndrome, coronary heart disease, non-small cell lung cancer, ulcerative colitis or proctitis, endometriosis, chronic otitis, benign prostatic hyperplasia, advanced brain tumor, bladder cancer, duodenal ulcers, metastatic breast cancer, prostate cancer, psoriasis, human uterine leiomyoma, gastric ulcer, discoid lupus erythematosus, lichen planus, colorectal cancer, acute myocardial infarction, advanced malignant glioma, endometrial cancer, malignant gliomas, head and neck or lung cancer, breast cancer, laryngeal and hypopharyngeal carcinoma, ovarian tumours, herpetic comeal ulcers and venous ulcers.

The invention will now be described with reference to the specific embodiments by means of the following Examples, which should not be construed as in any way limiting the present invention. The content of the description comprises all modifications and substitutions which can be practiced by a person skilled in the art in light of the above teachings and, therefore, with out extending beyond the meaning and purpose of the claims.

TABLE 1

| | | More Preferred |
|---|---|---|
| Amino Acid | Synonymous Groups | Synonymous Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

TABLE 2

| Amino Acid | Synonymous Groups |
|---|---|
| Ser | D-Ser, Thr, D-Thr, allo-Thr, Met, D-Met, Met(O), |
| | D-Met(O), L-Cys, D-Cys |
| Arg | D-Arg, Lys, D-Lys, homo-Arg, D-homo-Arg, Met, Ile, |
| | D-.Met, D-Ile, Orn, D-Orn |
| Leu | D-Leu, Val, D-Val, AdaA, AdaG, Leu, D-Leu, Met, D-Met |
| Pro | D-Pro, L-I-thioazolidine-4-carboxylic acid, |
| | D-or L-1-oxazolidine-4-carboxylic acid |
| Thr | D-Thr, Ser, D-Ser, allo-Thr, Met, D-Met, Met(O), |
| | D-Met(O), Val, D-Val |
| Ala | D-Ala, Gly, Aib, B-Ala, Acp, L-Cys, D-Cys |
| Val | D-Val, Leu, D-Leu, Ile, D-Ile, Met D-Met, AdaA, AdaG |
| Gly | Ala, D-Ala, Pro, D-Pro, Aib, .beta.-Ala, Acp |
| Ile | D-Ile, Val, D-Val, AdaA, AdaG, Leu, D-Leu, Met, D-Met |
| Phe | D-Phe, Tyr, D-Thr, L-Dopa, His, D-His, Trp, D-Trp, |
| | Trans-3, 4, or 5-phenylproline, AdaA, AdaG, cis-3, 4, |
| | or 5-phenylproline, Bpa, D-Bpa |
| Tyr | D-Tyr, Phe, D-Phe, L-Dopa, His, D-His |
| Cys | D-Cys, S—Me-Cys, Met, D-Met, Thr, D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, Met, D-Met, |
| | Ile, D-Ile, Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S—Me-Cys, Ile, D-Ile, Leu, D-Leu, Val, D-Val |

### EXAMPLES

### Example 1

Sequences of EGF protein domains from the ASTRAL database (Brenner S E et al “The ASTRAL compendium for protein structure and sequence analysis” Nucleic Acids Res. Jan. 1, 2000; 28 (1): 254-6) were used to search for homologous protein sequences in genes predicted from human genome sequence (Celera database). The protein sequences were obtained from the gene predictions and translations thereof as generated by one of three programs: the Genescan (Burge C, Karlin S., “Prediction of complete gene structures in human genomic DNA, J Mol Biol. Apr. 25, 1996; 268(1):78-94) Grail (Xu Y, Uberbacher E C., “Automated gene identification in large-scale genomic sequences”, J Comput Biol. 1997 Fall; 4(3):325-38) and Fgenesh (Proprietary Celera software).

The sequence profiles of the EGF domains were generated using PIMAII (Profile Induced Multiple Alignment; Boston University software, version 11, Das S and Smith T F 2000), an algorithm that aligns homologous sequences and generates a sequence profile. The homology was detected using PIMAII that generates global-local alignments between a query profile and a hit sequence. In this case the algorithm was used with the profile of the EGF functional domain as a query. PIMAII compares the query profile to the database of gene predictions translated into protein sequence and can therefore identify a match to a DNA sequence that contains that domain. Further comparison by BLAST (Basic Local Alignment Search Tool; NCBI version 2) of the sequence with known EFG containing proteins identified the closets homolog (Gish W, States D J. “Identification of protein coding regions by database similarity search.”, Nat Genet. March 1993; 3(3):266-72; Pearson W R, Miller W., “Dynamic programming algorithms for biological sequence comparison”, Methods Enzymol. 1992; 210:575-601; Altschul S F et al., “Basic local alignment search tool”, J Mol Biol. Oct. 5, 1990; 215(3):403-10). PIMAII parameters used for the detection were the PIMA prior amino acids probability matrix and a Z-cutoff score of 10. BLAST parameters used were: Comparison matrix=BLOSUM62; word length=3; E value cutoff=10; Gap opening and extension=default; No filter.

Once the functional domain was identified in the sequence, the genes were re-predicted with the genewise algorithm using the sequence of the closets homolog (Bimey E et al., “PairWise and SearchWise: finding the optimal alignment in a simultaneous comparison of a protein profile against all DNA translation frames”, Nucleic Acids Res. Jul. 15, 1996; 24(14):2730-9)

The profiles for homologous EGF domains were generated automatically using the PSI-BLAST (Altshul et al 1997) scripts written in PERL (Practical Extraction and Report Language) and PIMAII.

A total of 55 predicted genes out of the 464 matching the original query generated on the basis of EGF domain profiles were selected since they were judged as potentially novel.

The novelty of the protein sequences was finally assessed by searching protein databases (SwissProt/Trembl, Human IPI and Derwent GENESEQ) using BLAST and a specific annotation has been attributed on the basis of amino acid sequence homology.

### Example 2

One sequence isolated by the methodology set out in Example 1 is that referred to herein as the SCS0008 polypeptide sequence.

This sequence, even though it presents approximately 50% of homology with human Fibrillin 2 (as well as with other EGF domain containing proteins) in the central region, should be rather considered a human shorter splicing variant of a mouse gene coding for a different extracellular matrix protein recently cloned by two distinct groups and called nephronectin or POEM (see Morimura et al., J Biol Chem Nov. 9, 2001; 276(45):42172-81 ; Brandenberger et al., J Cell Biol Jul. 23, 2001; 154(2):447-58 (Comment in: J Cell Biol. Jul. 23, 2001; 154(2):257-9). This protein has been characterized so far as an adhesion molecule acting as a ligand for a(8)b(1) integrin and involved in the development and function of various organs, in particular kidney.

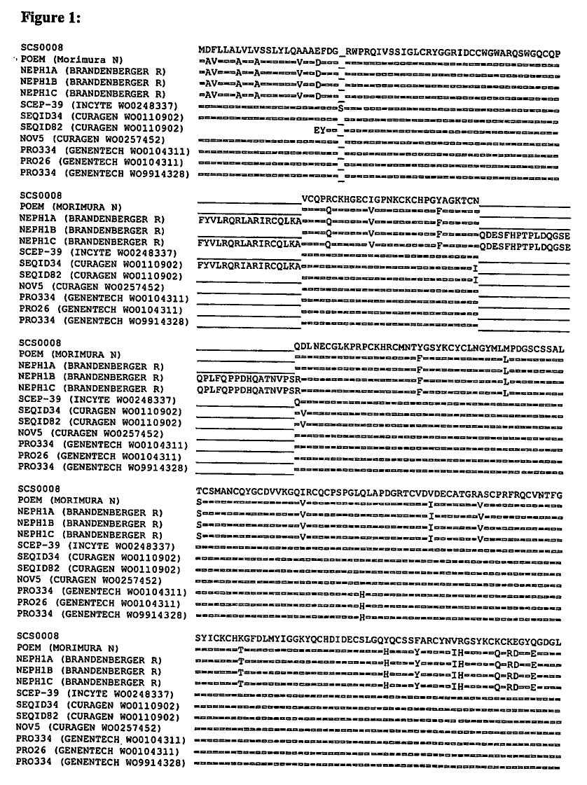

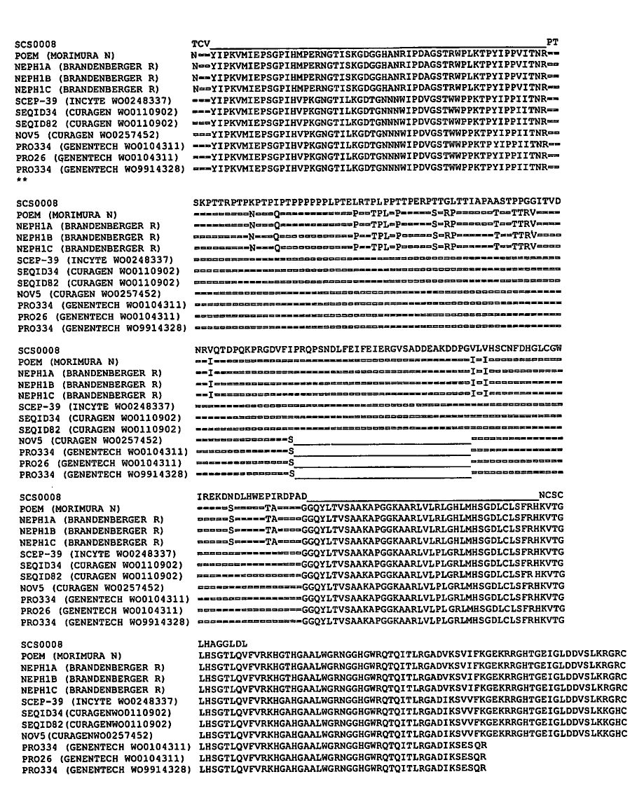

Larger Human variants have also been discovered (see SCEP-39 (INCYTE WO0248337); SEQID34 (CURAGEN WO0110902); SEQID82 (CURAGEN WO0110902); NOV5 (CURAGEN WO00257452); PRO334 (GENENTECH WO0104311); PRO26 (GENENTECH WO0104311); and PRO334 (GENENTECH WO9914328)), but none of them, as for the mouse sequences, corresponds to the submitted sequence which, in particular, lacks a central and the C-terminal region common to all relevant human/mouse sequence identified (see FIG. 1). The first sequence corresponds to a region located between the last EGF domain and a proline-rich region.

### Example 3

### Identification and Cloning of Splice Variants of SCS0008

3.1 Introduction:

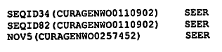

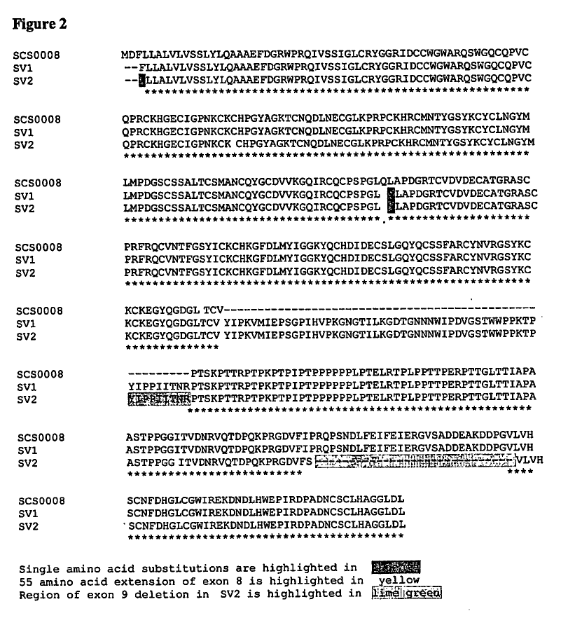

SCS0008 is a 1700 cDNA prediction encoding an EGF domain-containing protein of 406 amino acids with homology to fibrillin. Using nested PCR primers based on the full coding sequence of the SCS0008 prediction, two splice variants of SCS0008 from human lung (SVI) and human kidney (SV2) respectively. The alignments of SV1 and 2 with SCS0008 are shown in FIG. 2.

SV1 contains a 55 amino acid extension of exon 8 and a mutation at Q159H. SV2 also contains a 55 amino acid extension of exon 8, a mutation at Q159H but is missing exon 9. The cloned version of SV2 also contains a PCR induced mutation F3L, which was later corrected during subcloning. Both splice variants were subcloned with a C-terminal 6HIS tag using the Gateway cloning methodology into expression vectors pEAK12d and pDEST12.2.

3.2 Cloning of SCS0008-SV1

3.2.1 Preparation of Human Luna cDNA

First strand cDNA was prepared from normal lung total RNA (Clontech) using Superscript II RNase H⁻ Reverse Transcriptase (Invitrogen) according to the manufacturer's protocol. 1 μl Oligo (dT)₁₅ primer (500 μg/ml, Promega), 2 μg human lung total RNA, 1 μl of 10 mM dNTP mix (10 mM each of dATP,dGTP,dCTP and dTTP at neutral pH) and sterile distilled water to a final volume of 12 μl were combined in a 1.5 ml eppendorf tube, heated to 65° C. for 5 min and then chilled on ice. The contents were collected by brief centrifugation and 4 μl of 5× First-Strand Buffer, 2 μl of 0.1 M DTT, and 1 μl of RNaseOUT Recombinant Ribonuclease Inhibitor (40 units/μl, Invitrogen) were added. The contents of the tube were mixed gently and incubated at 42° C. for 2 min, then 1 μl (200 units) of SuperScript II enzyme was added and mixed gently by pipetting. The mixture was incubated at 42° C. for 50 min and then inactivated by heating at 70° C. for 15 min. To remove RNA complementary to the cDNA, 1 μl (2 units) of E. coli RNase H (Invitrogen) was added and the reaction mixture incubated at 37° C. for 20 min. The final 21 μl reaction mix was diluted by adding 179 μl sterile water to give a total volume of 200 μl.

3.2.2 Gene Specific Cloning Primers for PCR

A pair of PCR primers having a length of between 18 and 25 bases was designed for amplifying the complete coding sequence of the virtual cDNA using Primer Designer Software (Scientific & Educational Software, PO Box 72045, Durham, N.C. 27722-2045, USA). PCR primers were optimized to have a Tm close to 55±10° C. and a GC content of 40-60%. Primers were selected which had high selectivity for the target sequence (SCS0008) with little or no non-specific priming.

3.2.3 PCR Amplification of SCS0008 from Human Luna cDNA

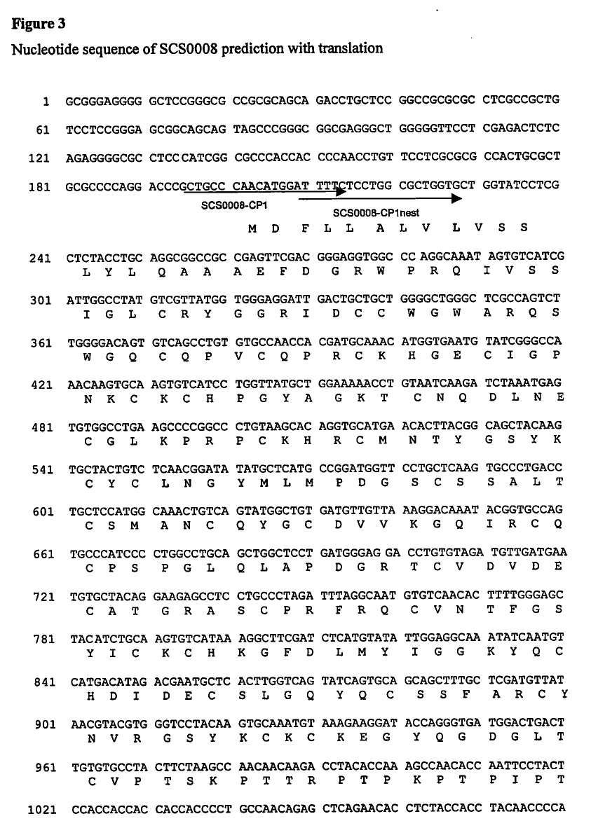

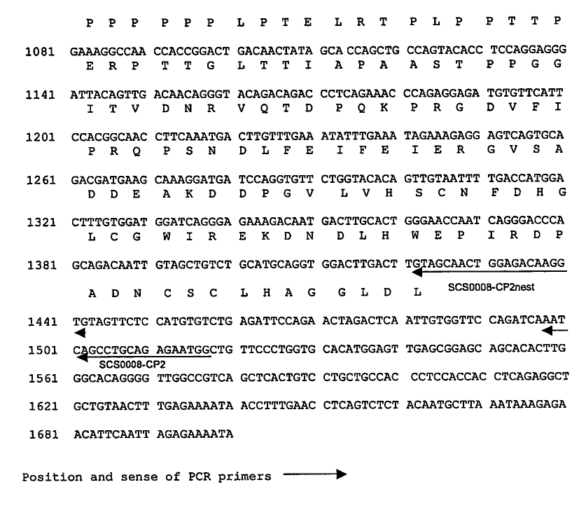

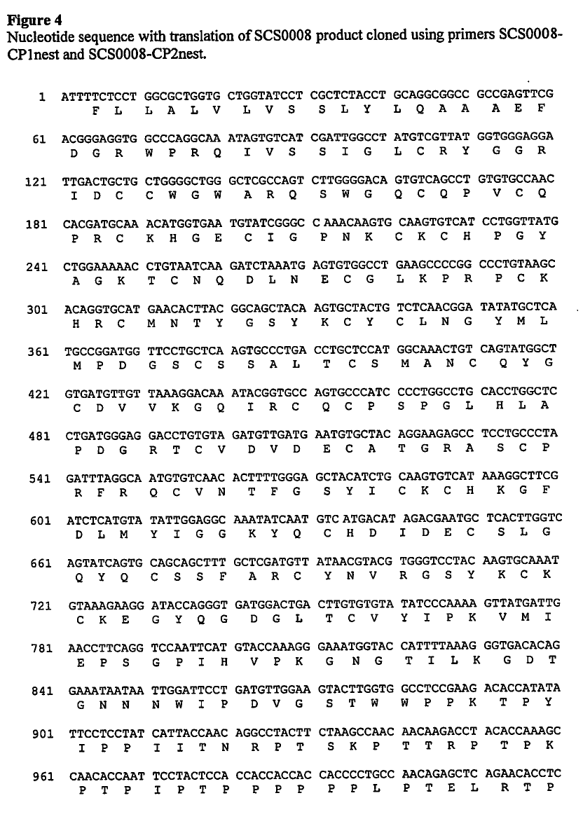

Gene-specific cloning primers (SCS0008-CP1 and SCS0008-CP2, FIG. 3, FIG. 4 and Table 3) were designed to amplify a cDNA fragment of 1323 bp covering the entire coding sequence of the SCS0008 prediction. The gene-specific doning primers SCS0008-CP1 and SCS0008-CP2 were used with human lung cDNA as the template. The PCR was performed in a final volume of 50 μl containing 1× AmpliTaq™ buffer, 200 μM dNTPs, SCS0008-CP1, 50 pmoles of SCS0008-CP2, 2.5 units of AmpliTaq™ (Perkin Elmer) and 100 ng of lung cDNA using an MJ Research DNA Engine, programmed as follows: 94° C., 2 min; 40 cycles of 94° C., 1 min, 50° C., 1 min, and 72° C., 1 min; followed by 1 cycle at 72° C. for 7 min and a holding cycle at 4° C. PCR products were purified directly using the Wizard PCR Preps DNA Purification System (Promega). PCR products were eluted in 50 μl of sterile water and 10 μl of the amplifcation products were then used as template in a 2^{nd} PCR reaction using the same conditions as described above except that the primers used were nested primers SCS0008-CP1 nest and SCS0008 -CP2 nest. Amplification products were visualized on 0.8% agarose gels in 1×TAE buffer (Invitrogen) and a single PCR product migrating near the predicted molecular mass was purified from the gel using the Wizard PCR Preps DNA Purification System (Promega). The PCR product was eluted in 50 μl of sterile water and subcloned directly.

3.2.4 Subclonina of PCR Products

PCR products were subcloned into the topoisomerase I modified cloning vector (pCR4-TOPO) using the TOPO cloning kit purchased from the Invitrogen Corporation using the conditions specified by the manufacturer. Briefly, 4 μl of gel purified PCR product was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm SOC media (room temperature) was added. Samples were incubated with shaking (220 rpm) for 1 h at 37° C. The transformation mixture was then plated on L-broth (LB) plates containing ampicillin (100 μg/ml) and incubated overnight at 37° C. Ampicillin resistant colonies containing inserts were identified by colony PCR.

3.2.5 Colony PCR

Colonies were inoculated into 50 μl sterile water using a sterile toothpick. A 10 μl aliquot of the inoculum was then subjected to PCR in a total reaction volume of 20 μl as described above, except the primers used were T7 and T3. The cycling conditions were as follows: 94° C., 2 min; 30 cycles of 94° C., 30 sec, 48° C., 30 sec and 72° C., 1 min. Samples were then maintained at 4° C. (holding cycle) before further analysis.

PCR reaction products were analyzed on 1% agarose gel in 1×TAE buffer. Colonies which gave the expected PCR product size (+105 bp due to the multiple cloning site or MCS) were grown up overnight at 37° C. in 5 ml L-Broth (LB) containing ampicillin (100 μg/ml), with shaking (220 rpm).

3.2.6 Plasmid DNA Preparation and Sequencing

Miniprep plasmid DNA was prepared from the 5 ml culture using a Qiaprep Turbo 9600 robotic system (Qiagen) or Wizard Plus SV Minipreps kit (Promega cat. no. 1460) according to the manufacturer's instructions. Plasmid DNA was eluted In 100 μl of sterile water. The DNA concentration was measured using an Eppendorf BO photometer. Plasmid DNA (200-500 ng) was subjected to DNA sequencing with the T7 primer and SP6 primer using the Big Dye Terminator system (Applied Biosystems cat no. 4390246) according to the manufacturers instructions. The primer sequences are shown in Table 3. Sequencing reactions were purified using Dye-Ex columns (Qiagen) or Montage SEQ 96 cleanup plates (Millipore cat no. LSKS09624) then analyzed on an Applied Biosystems 3700 sequencer.

Sequence analysis of the cloned cDNA insert revealed a match to the predicted SCS0008 sequence over exons 1-8 except for a single point mutation at nucleotide 681 which lead to an amino acid substitution Q159H. There was also a 165 bp insertion at the end of exon 8 leading to an in frame insertion of 55 amino acids detected in all clones sequenced. The sequence of the cloned cDNA fragment is shown in FIG. 4.

3.3 Construction of Mammalian Cell Expression Vectors for SCS00008-SV1

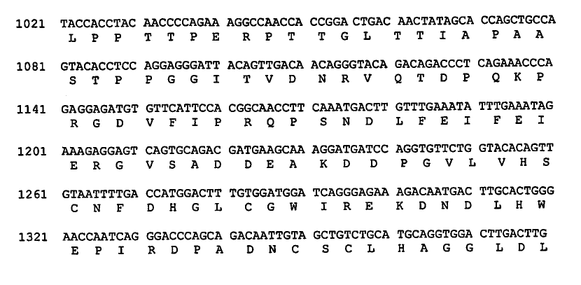

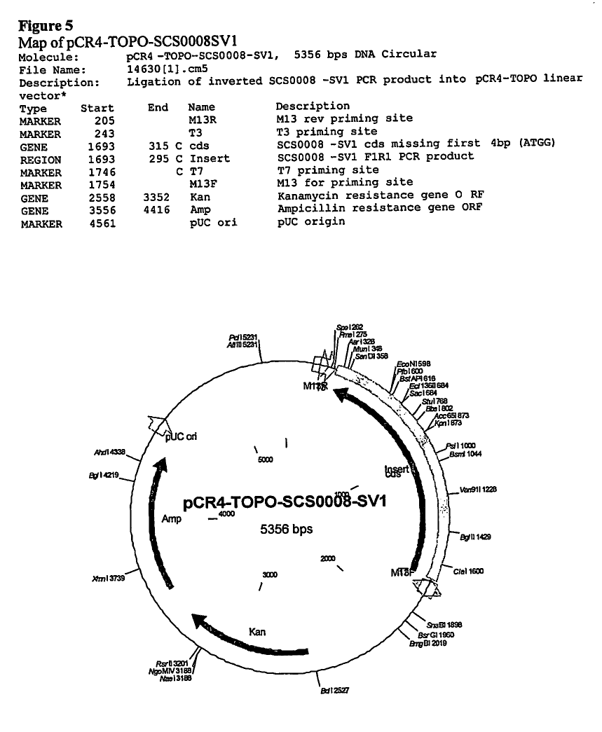

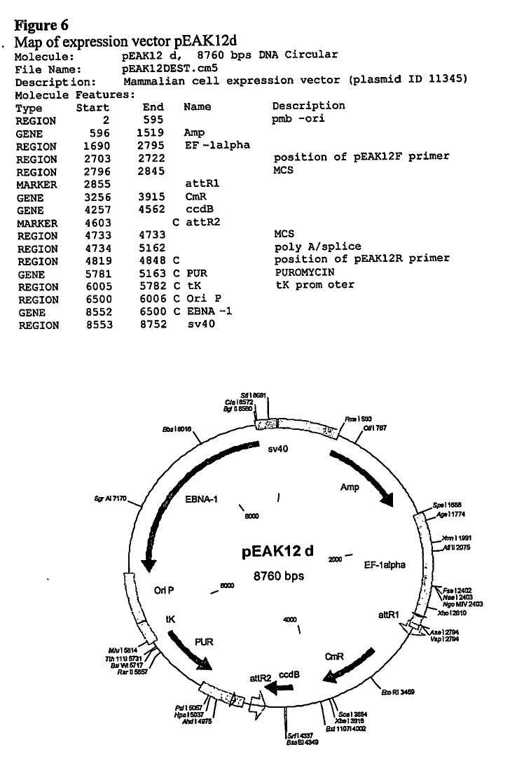

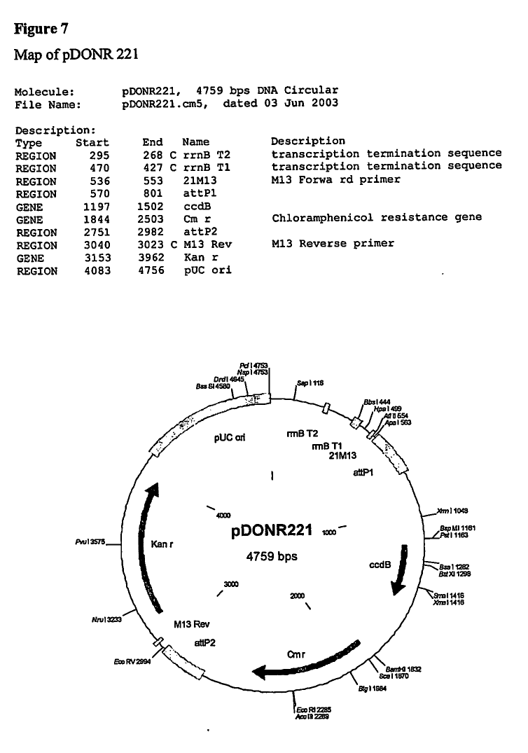

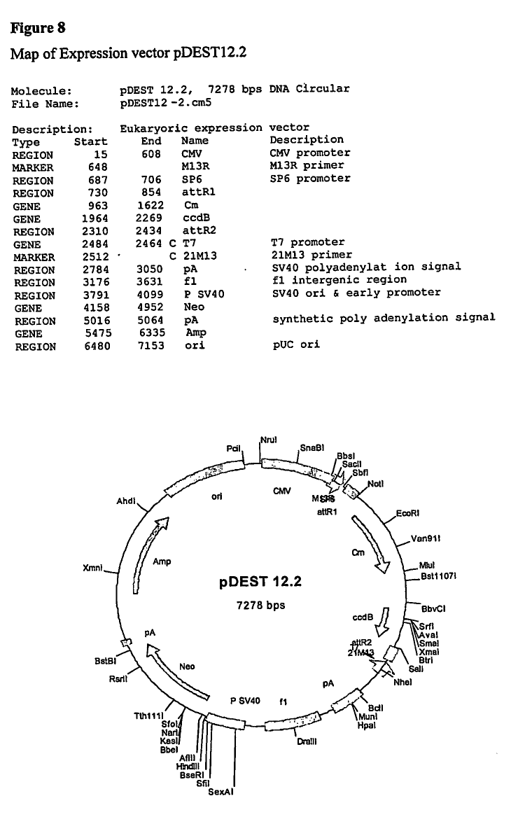

A pCR4-TOPO clone containing the coding sequence (ORF) of SCS0008 -SV1 identified by DNA sequencing (pCR4-TOPO-SCS0008SV1, plasmid ID. 14630) (FIG. 5) was then used to subclone the insert into the mammalian cell expression vectors pEAK12d (FIG. 6) and pDEST12.2 (FIG. 8) using the Gateway™ cloning methodology (Invitrogen).

3.3.1 Generation of Gateway Compatible SCS0008-SV1 ORF Fused to an in Frame 6HIS Tag Sequence.

The first stage of the Gateway cloning process involves a two step PCR reaction which generates the ORF of SCS0008-SV1 flanked at the 5′ end by an attB1 recombination site and Kozak sequence, and flanked at the 3′ end by a sequence encoding an in frame 6 histidine (6HIS) tag, a stop codon and the attB2 recombination site (Gateway compatible cDNA). The first PCR reaction (in a final volume of 50 μl) contains: 1 μl (40 ng) of pCR4-TOPO-SCS0008-SV1 (plasmid ID 14630), 1.5 μl dNTPs (10 mM), 10 μl of 10×Pfx polymerase buffer, 1 μl MgSO4 (50 mM), 0.5 μl each of gene specific primer (100 μM) (SCS0008-SV1-EX1 and SCS0008-SV1-EX2), 2.5 μl 10× Enhancer™ solution (Invitrogen) and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification products were visualized on 0.8% agarose gel in 1×TAE buffer (Invitrogen) and a product migrating at the predicted molecular mass was purified from the gel using the Wizard PCR Preps DNA Purification System (Promega) and recovered in 50 μl sterile water according to the manufacturer's instructions.

The second PCR reaction (in a final volume of 50 μl) contained 10 μl purified PCR 1 product, 1.5 μl dNTPs (10 mM), 5 μl of 10×Pfx polymerase buffer, 1 μl MgSO4 (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR products were gel purified using the Wizard PCR prep DNA purification system (Promega) according to the manufacturer's instructions.

3.3.2 Subcloninq of Gateway Compatible SCS0008-SV1 ORF into Gateway Entry Vector pDONR221 and Expression Vectors pEAK12d and pDEST12.2

The second stage of the Gateway cloning process involves subcloning of the Gateway modified PCR product into the Gateway entry vector pDONR221 (Invitrogen, FIG. 7) as follows: 5 μl of purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of proteinase K 1 μl (2 μg/μl) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml), which had been pre-warmed to room temperature, was added immediately after electroporation. The mixture was transferred to a 15 ml snap-cap tube and incubated, with shaking (220 rpm) for 1 h at 37° C. Aliquots of the transformation mixture (10μl and 50 μl) were then plated on L-broth (LB) plates containing kanamycin (40 μg/ml) and incubated overnight at 37° C.

Plasmid mini-prep DNA was prepared from 5 ml cultures from 6 of the resultant colonies using a Qiaprep Turbo 9600 robotic system (Qiagen). Plasmid DNA (150-200 ng) was subjected to DNA sequencing with 21M13 and M13Rev primers using the BigDyeTerminator system (Applied Biosystems cat no. 4390246) according to the manufacturer's instructions. The primer sequences are shown in Table 3. Sequencing reactions were purified using Dye-Ex columns (Qiagen) or Montage SEQ 96 cleanup plates (Millipore cat. no. LSKS09624) then analyzed on an Applied Biosystems 3700 sequencer.

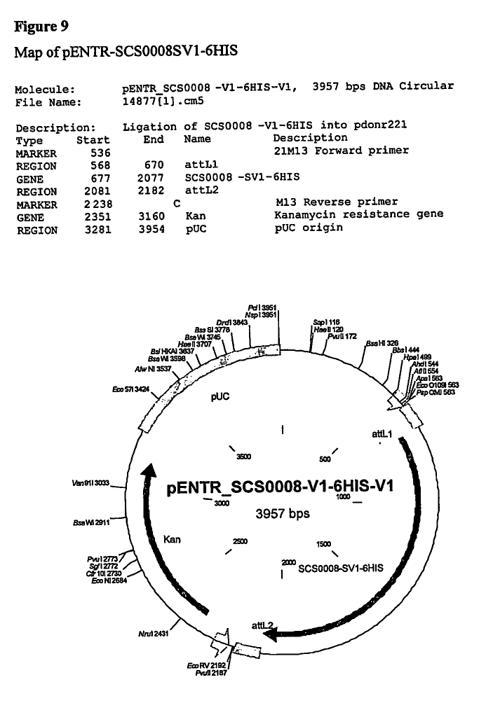

Plasmid eluate (2 μl or approx. 150 ng) from one of the clones which contain ed the correct sequence (pENTR-SCS0008-SV1-6HIS, plasmid ID 14877, FIG. 9) was then used in a recombination reaction containing 1.5 μl of either pEAK12d vector or pDEST12.2 vector (FIGS. 4 & 5) (0.1 μg/μl), 2 μl LR buffer and 1.5 μl of LR clonase (Invitrogen) in a final volume of 10 μl. The mixture was incubated at RT for 1 h, stopped by addition of proteinase K (1 μl at 2 μg/μl) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml), which had been pre-warmed to room temperature, was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F, pEAK12R, SCS0008SV1-SP1 and SCS0008SV1-SP2 primers as described above. Plasmid DNA (200-500 ng) in the pDEST12.2 vector was subjected to DNA sequencing with 21M13 and M13Rev, SCS0008SV1-SP1 and SCS0008SV1-SP2 primers as described above. Primer sequences are shown in Table 3

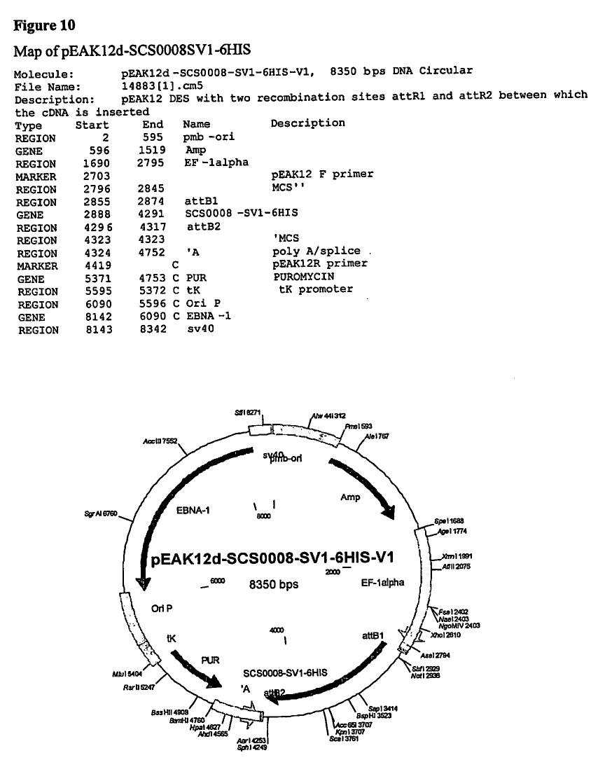

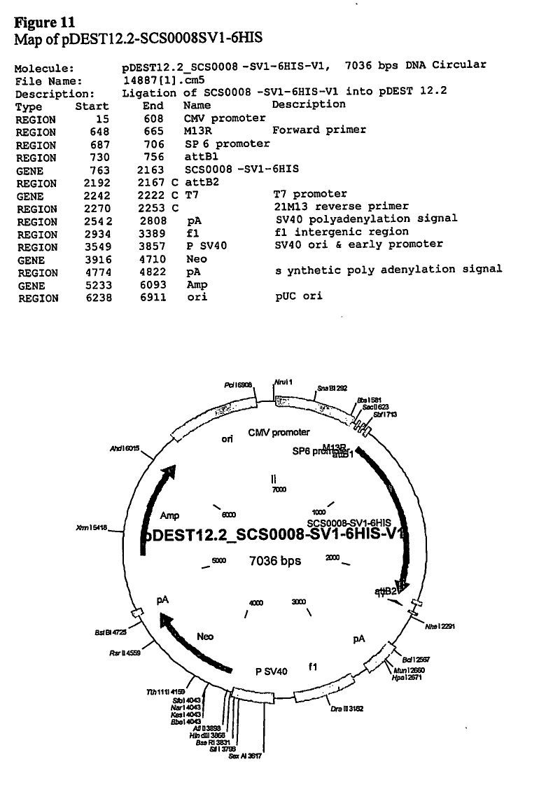

CsCl gradient purified maxi-prep DNA was prepared from a 500 ml culture of one of each of the sequence verified clones (pEAK12d-SCS0008-SV1-6HIS, plasmid ID number 14883, FIG. 10, and pDEST12.2-SCS0008-SV1-6HIS, plasmid ID 14887, FIG. 11) using the method described by Sambrook J. et al., 1989 (in Molecular Cloning, a Laboratory Manual, 2^{nd} edition, Cold Spring Harbor Laboratory Press), Plasmid DNA was resuspended at a concentration of 1 μl/μl in sterile water (or 10 mM Tris-HCl pH 8.5) and stored at −20° C.

TABLE 3

SCS0008SV1 cloning and sequencing primers

| | |
|---|---|
| Primer | Sequence (5′-3′) |

| | |
|---|---|
| SCS0008-CP1 | GCT GCC CAA CAT GGA TTT TC |
| SCS0008-CP2 | CCA TTC TCT GCA GGC TGA TT |
| SCS0008-CP1nest | ATT TTC TCC TGG CGC TGG TG |
| SCS0008-CP2nest | CACCTTGTCTCCAGTTGCTACA |
| SCS0008SV1-EX1 | AA GCA GGC TTC GCC ACC ATG GAT TTT |
| | CTC CTG GCG CTG GTG |
| SCS0008SV1-EX2 | GTG ATG GTG ATG GTG CAA GTC AAG |
| | TCC ACC TGC AT |
| GCP Forward | G GGG ACA AGT TTG TAC AAA AAA GCA |
| | GGC TTC GCC ACC |
| GCP Reverse | GGG GAC CAC TTT GTA CAA GAA AGC |
| | TGG GTT TCA ATG GTG ATG GTG ATG |
| | GTG |
| SCS0008SV1-SP1 | ATA CGG TGC CAG TGC CCA TC |
| SCS0008SV1-SP2 | CAA CAG GCC TAC TTC TAA GC |
| pEAK12F | GCC AGC TTG GCA CTT GAT GT |
| pEAK12R | GAT GGA GGT GGA CGT GTC AG |
| 21M13 | TGT AAA ACG ACG GCC AGT |
| M13REV | CAG GAA ACA GCT ATG ACC |
| T7 primer | TAA TAC GAC TCA CTA TAG GG |
| SP6 primer | ATT TAG GTG ACA CTA TAG |

Underlined sequence = Kozak sequence
Bold = Stop codon
Italic sequence = His tag

3.4 Cloning of SCS0008-SV2

3.4.1 Preparation of Human Kidney cDNA

First strand cDNA was prepared from normal kidney total RNA (Clontech) using Superscript II RNase H⁻ Reverse Transcriptase (Invitrogen) according to the manufacturer's protocol. 1 μl Oligo (dT)₁₅ primer (500 μg/ml, Promega), 2 μg human lung total RNA, 1 μl of 10 mM dNTP mix (10 mM each of dATP,dGTP,dCTP and dTTP at neutral pH) and sterile distilled water to a final volume of 12 μl were combined in a 1.5 ml eppendorf tube, heated to 65° C. for 5 min and then chilled on ice. The contents were collected by brief centrifugation and 4 μl of 5× First-Strand Buffer, 2 μl of 0.1 M DTT, and 1 μl of RNaseOUT Recombinant Ribonuclease Inhibitor (40 units/∥l, Invitrogen) were added. The contents of the tube were mixed gently and incubated at 42° C. for 2 min, then 1 μl (200 units) of SuperScript II enzyme was added and mixed gently by pipetting. The mixture was incubated at 42° C. for 50 min and then inactivated by heating at 70° C. for 15 min. To remove RNA complementary to the cDNA, 1 μl (2 units) of E. coli RNase H (Invitrogen) was added and the reaction mixture incubated at 37° C. for 20 min. The final 21 μl reaction mix was diluted by adding 179 μl sterile water to give a total volume of 200 μl.

3.4.2 Gene Specific Cloning Drimers for PCR

A pair of PCR primers having a length of between 18 and 25 bases was designed for amplifying the complete coding sequence of the virtual cDNA using Primer Designer Software (Scientific & Educational Software, PO Box 72045, Durham, N.C. 27722-2045, USA). PCR primers were optimized to have a Tm close to 55±10° C. and a GC content of 4060%. Primers were selected which had high selectivity for the target sequence (SCS00008) with little or no none specific priming.

3.4.3 PCR Amplification of SCS0008 from Human Kidney cDNA

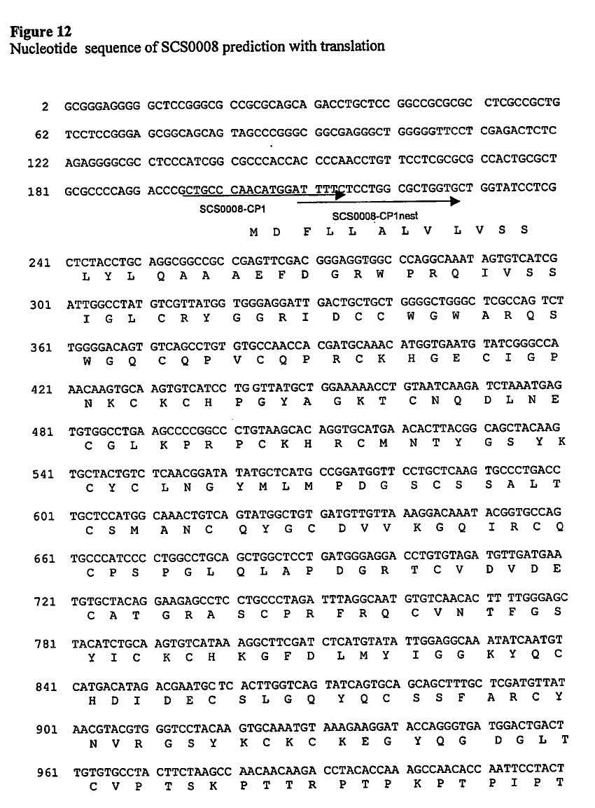

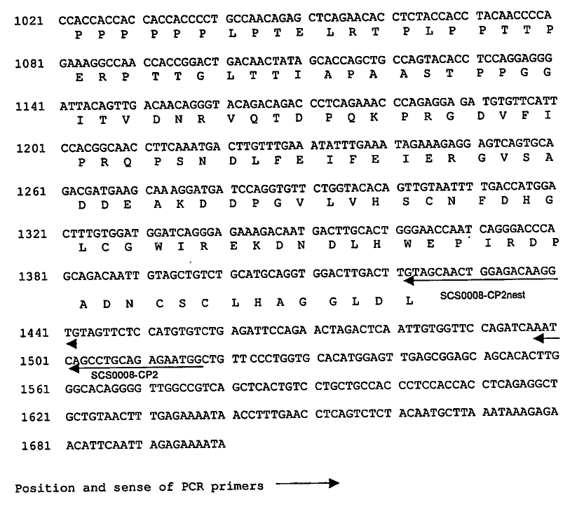

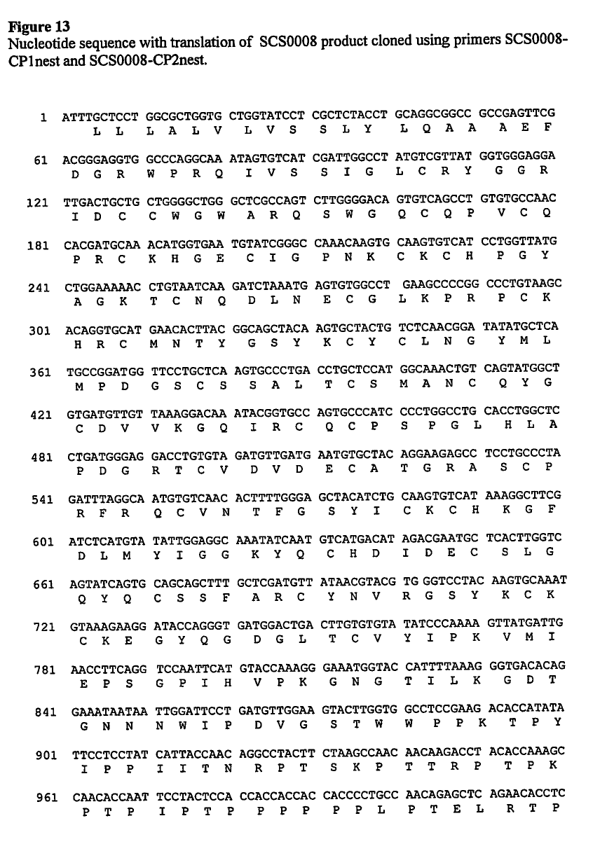

Gene-specific cloning primers (SCS0008-CP1 and SCS0008-CP2, FIG. 12, FIG. 13 and Table 4) were designed to amplify a cDNA fragment of 1323 covering the entire coding sequence of the SCS0008 prediction. The gene-specific cloning primers SCS0008-CP1 and SCS0008-CP2 were used with human kidney cDNA as the template. The PCR was performed in a final volume of 50 μl containing 1× AmpliTaq™ buffer, 200 μM dNTPs, SCS0008-CP1, 50 pmoles of SCS0008-CP2, 2.5 units of AmpliTaq™ (Perkin Elmer) and 100 ng of lung cDNA using an MJ Research DNA Engine, programmed as follows: 94° C., 2 min; 40 cycles of 94° C., 1 min, 50° C., 1 min, and 72° C., 1 min; followed by 1 cycle at 72° C. for 7 min and a holding cycle at 4° C. PCR products were purified directly using the Wizard PCR Preps DNA Purification System (Promega). PCR products were eluted in 50 μl of sterile water and 10 μl of the amplification products were then used as template in a 2^{nd} PCR reaction using the same conditions as described above except that the primers used were nested primers SCS0008-CP1 nest and SCS0008-CP2 nest. Amplification products were visualized on 0.8% agarose gels in 1×TAE buffer (Invitrogen) and a single PCR product migrating near the predicted molecular mass was purified from the gel using the Wizard PCR Preps DNA Purification System (Promega). The PCR product was eluted in 50 μl of sterile water and subcloned directly.

3.4.4 Subcloning of PCR Products

PCR products were subcloned into the topoisomerase I modified cloning vector (pCR4-TOPO) using the TOPO cloning kit purchased from the Invitrogen Corporation using the conditions specified by the manufacturer. Briefly, 4 μl of gel purified PCR product was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm SOC media (room temperature) was added. Samples were incubated with shaking (220 rpm) for 1 h at 37° C. The transformation mixture was then plated on L-broth (LB) plates containing ampicillin (100 μg/ml) and incubated overnight at 37° C. Ampicillin resistant colonies containing inserts were identified by colony PCR.

3.4.5 Colony PCR

Colonies were inoculated into 50 μl sterile water using a sterile toothpick. A 10 μl aliquot of the inoculum was then subjected to PCR in a total reaction volume of 20 μl as described above, except the primers used were T7 and T3. The cycling conditions were as follows: 94° C., 2 min; 30 cycles of 94° C., 30 sec, 48° C., 30 sec and 72° C., 1 min. Samples were then maintained at 4° C. (holding cycle) before further analysis.

PCR reaction products were analyzed on 1% agarose gel in 1×TAE buffer. Colonies which gave the expected PCR product size (+105 bp due to the multiple cloning site or MCS) were grown up overnight at 37° C. in 5 ml L-Broth (LB) containing ampicillin (100 μg/ml), with shaking (220 rpm).

3.4.6 Plasmid DNA Preparation and Sequencing

Miniprep plasmid DNA was prepared from the 5 ml culture using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an

Eppendorf BO photometer. Plasmid DNA (200-500 ng) was subjected to DNA sequencing with the T7 primer and SP6 primer using the BigDyeTerminator system (Applied Biosystems cat no. 4390246) according to the manufacturer's instructions. The primer sequences are shown in Table 4. Sequencing reactions were purified using Dye-Ex columns (Qiagen) or Montage SEQ 96 deanup plates (Millipore cat. no. LSKS09624) then analyzed on an Applied Biosystems 3700 sequencer.

Sequence analysis of the cloned cDNA insert revealed a match to the predicted SCS0008 sequence over exons 1-8 except for single point mutations at nucleotide 213 (T to G) which lead to an amino acid substitution of F3L, and at nucleotide 681 (G to C) which lead to an amino acid substitution Q159H. There was also a 165 bp insertion at the end of exon 8 leading to an in frame insertion of 55 amino acids detected in all clones sequenced. In 3/9 clones sequenced an 87 bp deletion (corresponding to exon 9) was also detected. These 3 clones likely represent a splice variant of SCS0008 (SCS0008-SV2) which lacks exon 9. The sequence of the cloned SCS0008-SV2 cDNA fragment is shown in FIG. 13.

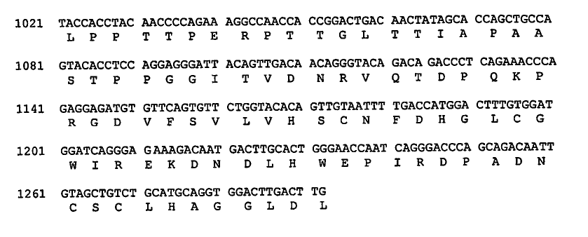

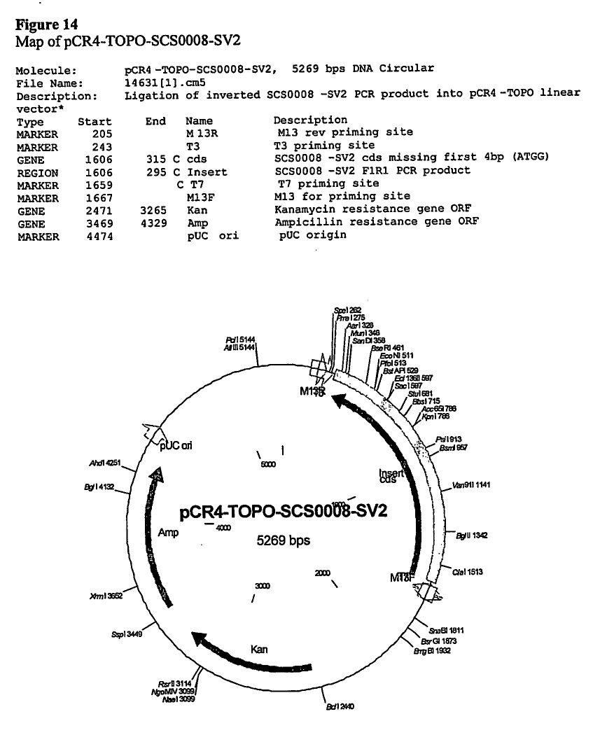

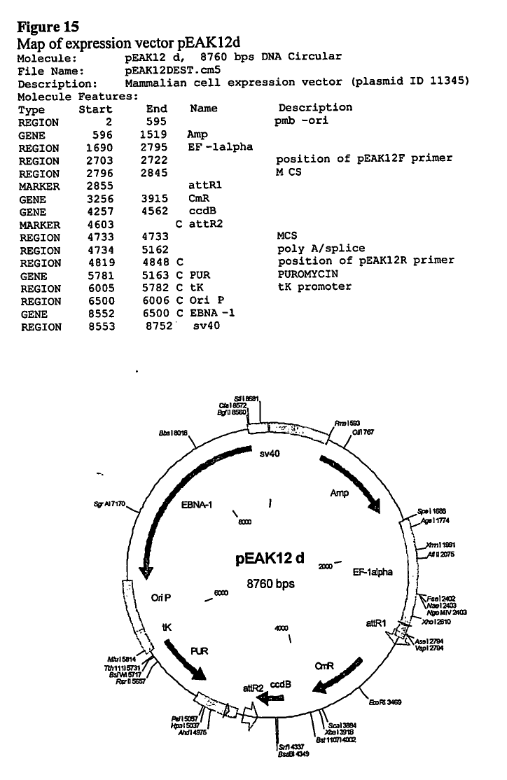

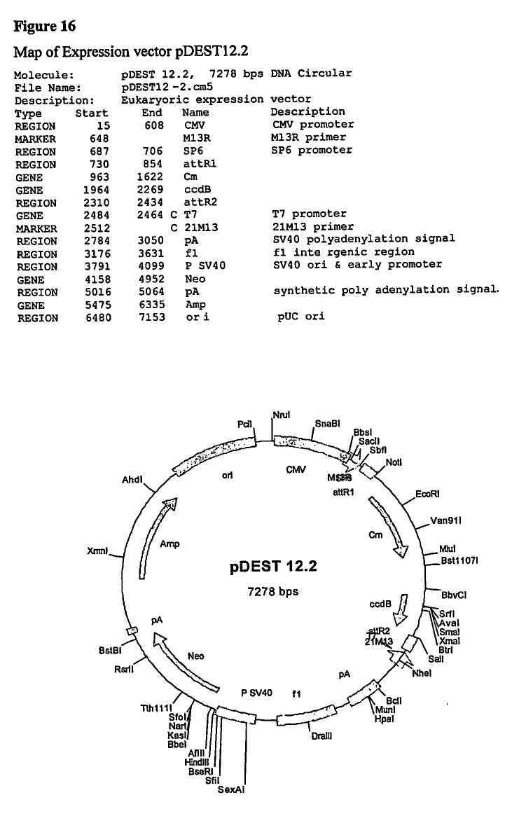

3.5 Construction of mammalian cell exDression vectors for SCS00008 -SV2 A pCR4-TOPO clone containing the coding sequence (ORF) of SCS0008-SV2 identified by DNA sequencing (pCR4-TOPO-SCS0008SV2, plasmid ID. 14631) (FIG. 14) was then used to subclone the insert into the mammalian cell expression vectors pEAK12d (FIG. 15) and pDEST12.2 (FIG. 16) using the Gateway™ cloning methodology (Invitrogen).

3.5.1 Generation of Gateway Compatible SCS0008-SV2 ORF Fused to an in Frame 6HIS Tag Sequence.

The first stage of the Gateway cloning process involves a two step PCR reaction which generates the ORF of SCS0008-SV2 flanked at the 5′ end by an attBl recombination site and Kozak sequence, and flanked at the 3′ end by a sequence encoding an in frame 6 histidine (6HIS) tag, a stop codon and the aftB2 recombination site (Gateway compatible cDNA). The first PCR reaction (in a final volume of 50 μl) contains: 1 μl (40 ng) of pCR4-TOPO-SCS0008-SV2 (plasmid ID 14631), 1.5 μl dNTPs (10 mM), 10 μl of 10×Pfx polymerase buffer, 1 μl MgSO4 (50 mM), 0.5 μl each of gene specific primer (100 μM) (SCS0008-SV2-EX1 and SCS0008-SV2-EX2), 2.5 μl 10× Enhancer™ solution (Invitrogen) and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification products were visualized on 0.8% agarose gel in 1×TAE buffer (Invitrogen) and a product migrating at the predicted molecular mass was purified from the gel using the Wizard PCR Preps DNA Purification System (Promega) and recovered in 50 μl sterile water according to the manufacturer's instructions.

The second PCR reaction (in a final volume of 50 μl) contained 10 μl purified PCR 1 product, 1.5 μl dNTPs (10 mM), 5 μl of 10×Pfx polymerase buffer, μl MgSO4 (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR products were gel purified using the Wizard PCR prep DNA purification system (Promega) according to the manufacturer's instructions.

3.5.2 Subcloning of Gateway Compatible SCS0008-SV2 ORF into Gateway Entry Vector pDONR221 and Expression Vectors pEAK12d and pDEST12.2

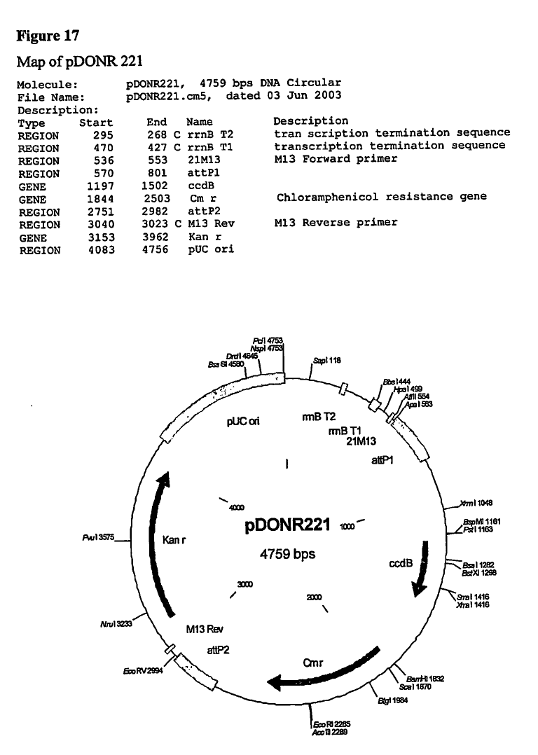

The second stage of the Gateway cloning process involves subcloning of the Gateway modified PCR product into the Gateway entry vector pDONR221 (Invitrogen, FIG. 17) as follows: 5 μl of purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of proteinase K 1 μl (2 μg/μL) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml), which had been pre-warmed to room temperature, was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight at 37° C.

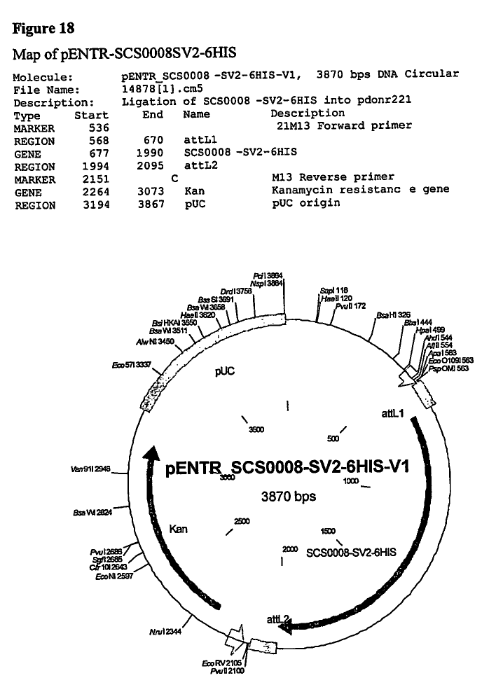

Plasmid mini-prep DNA was prepared from 5 ml cultures from 6 of the resultant colonies using a Qiaprep Turbo 9600 robotic system (Qiagen). Plasmid DNA (150-200 ng) was subjected to DNA sequencing with 21M13 and M13Rev primers using the BigDyeTerminator system (Applied Biosystems cat. no. 4390246) according to the manufacturer's instructions. The primer sequences are shown in Table 4. Sequencing reactions were purified using Dye-Ex columns (Qiagen) or Montage SEQ 96 cleanup plates (Millipore cat. no. LSKS09624) then analyzed on an Applied Biosystems 3700 sequencer. Plasmid eluate (2·μli or approx. 150 ng) from one of the clones which contain ed the correct sequence (pENTR-SCS0008-SV2-6HIS, plasmid ID 14878, FIG. 18) was then used in a recombination reaction containing 1.5 μl of either pEAK12d vector or pDEST12.2 vector (FIGS. 4 & 5) (0.1 μg/μl), 2 μl LR buffer and 1.5 μl of LR clonase (Invitrogen) in a final volume of 10 μl. The mixture was incubated at RT for 1 h, stopped by addition of proteinase K (1 μl at 2 μg/μl) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvefte and the cells electroporated using a BioRad Gene-Pulser™ according to the manufacturer's recommended protocol. SOC media (0.5 ml), which had been pre-warmed to room temperature, was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F, pEAK12R, SCS0008SV1-SP1 and SCS0008SV1-SP2 primers as described above. Plasmid DNA (200-500 ng) in the pDEST12.2 vector was subjected to DNA sequencing with 21M13 and M13Rev, SCS0008SV1-SP1 and SCS0008SV1-SP2 primers as described above. Primer sequences are shown in Table 4.

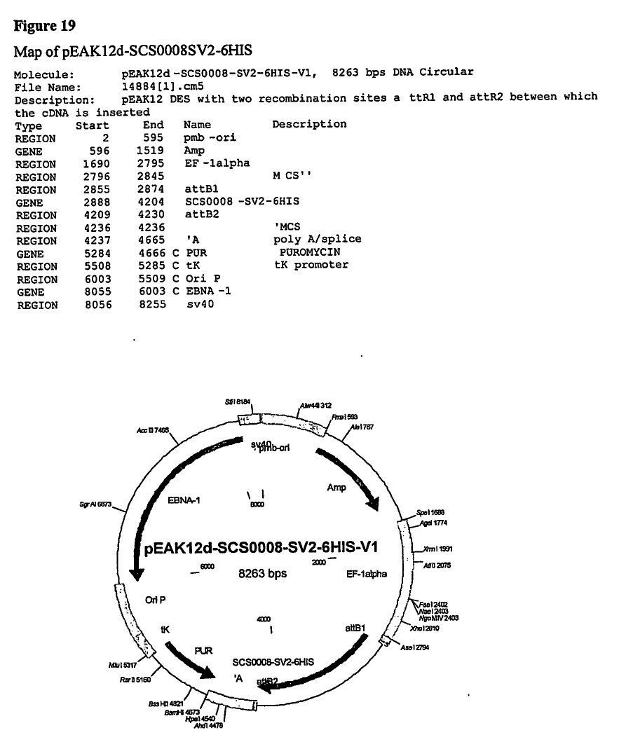

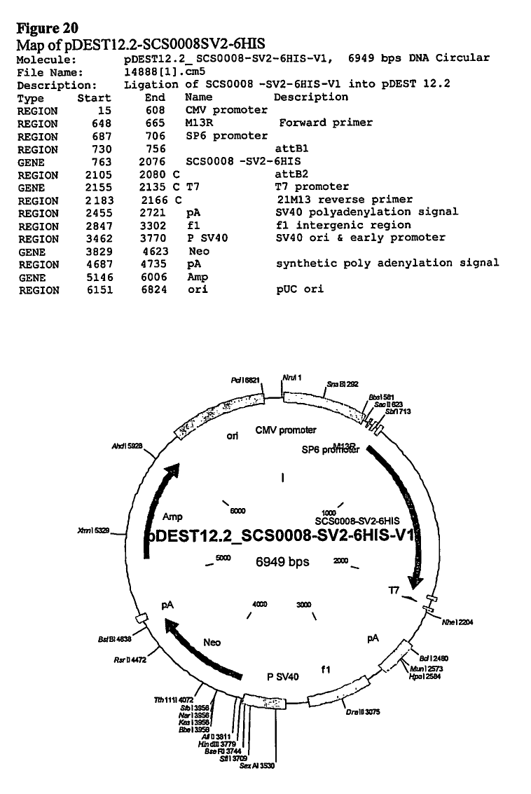

CsCl gradient purified maxi-prep DNA was prepared from a 500 ml culture of one of each of the sequence verified clones (pEAK12d-SCS0008-SV2-6HIS, plasmid ID number 14884, FIG. 19, and pDEST12.2-SCS0008-SV2-6HIS, plasmid ID 14888, FIG. 20) using the method described by Sambrook J. et al., 1989 (in Molecular Cloning, a Laboratory Manual, 2^{nd} edition, Cold Spring Harbor Laboratory Press), Plasmid DNA was resuspended at a concentration of 1 μg/μl in sterile water (or 10 mM Tris-HCl pH 8.5) and stored at −20° C.

TABLE 4

SCS0008-SV2 cloning and sequencing primers

| | |
|---|---|
| Primer | Sequence (5′-3′) |

| | |
|---|---|
| SCS0008-CP1 | GCT GCC CAA CAT GGA TTT TC |
| SCS0008-CP2 | CCA TTC TCT GCA GGC TGA TT |
| SCS0008-CP1nest | ATT TTC TCC TGG CGC TGG TG |
| SCS0008-CP2nest | CACCTTGTCTCCAGTTGCTACA |
| SCS0008SV2-EX1 | AA GCA GGC TTC GCC ACC ATG GAT TTT |
| | CTC CTG GCG CTG GTG |
| SCS0008SV2-EX2 | GTG ATG GTG ATG GTG CAA GTC AAG |
| | TCC ACC TGC AT |
| GCP Forward | G GGG ACA AGT TTG TAC AAA AAA GCA |
| | GGC TTC GCC ACC |
| GCP Reverse | GGG GAC CAC TTT GTA CAA GAA AGC |
| | TGG GTT TCA ATG GTG ATG GTG ATG |
| | GTG |
| SCS0008SV1-SP1 | ATA CGG TGC CAG TGC CCA TC |
| SCS0008SV1-SP2 | CAA CAG GCC TAC TTC TAA GC |
| pEAK12F | GCC AGC TTG GCA CTT GAT GT |
| pEAK12R | GAT GGA GGT GGA CGT GTC AG |
| 21M13 | TGT AAA ACG ACG GCC AGT |
| M13REV | CAG GAA ACA GCT ATG ACC |
| T7 primer | TAA TAC GAC TCA CTA TAG GG |
| SP6 primer | ATT TAG GTG ACA CTA TAG |

Underlined sequence = Kozak sequence
Bold = Stop codon
Italic sequence = His tag

### Example 4

### Identification and Description of SCS0008 Domains

4.1 Identification

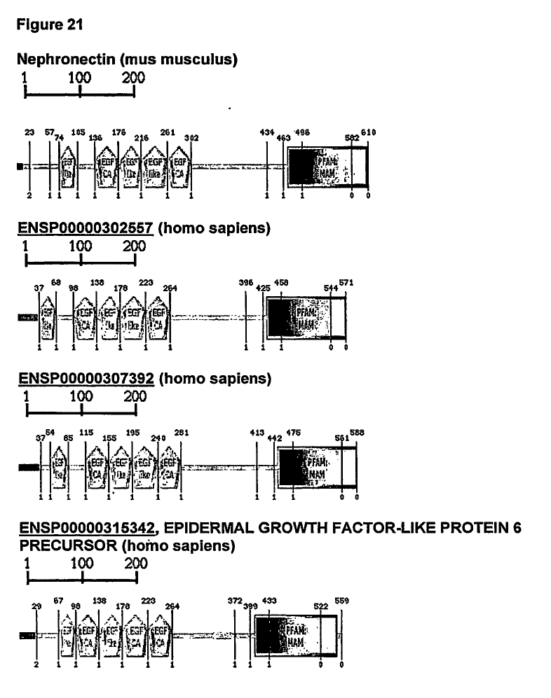

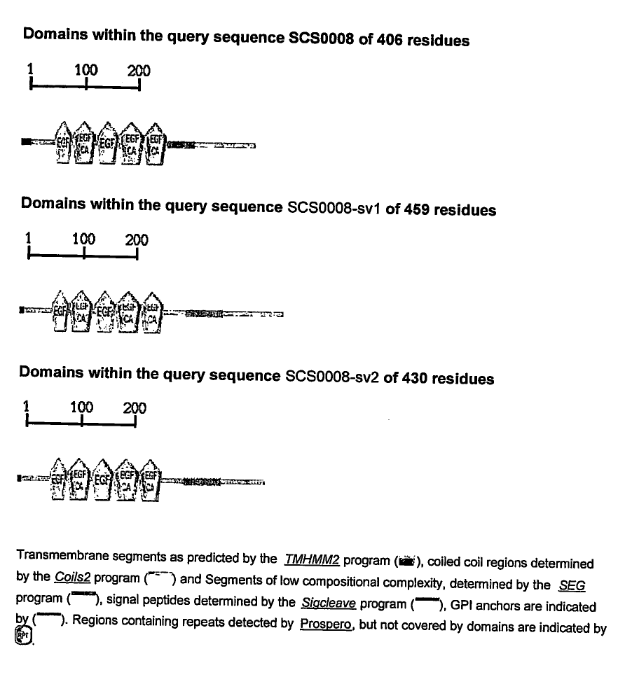

A bioinformatic tool called SMART (http://smart.embl-heidelberg.de/) was used to identify the putative domains of SCS0008 and of the splice variants SCS0008-SV1 and SCS0008-SV2. Results are shown in FIG. 21, which also display the mouse ortholog nephronectin, other known human splice variants of SCS0008, and one protein displaying similar domain organization, i.e. EGFL6. In addition, Prosite was also run on the sequences (http://us.expasy.org/prosite/).

TABLE 5
Domains identified by SMART within
the query sequence SCS0008 of 406 residues
Confidently predicted domains, repeats, motifs and features:

| name | begin | end | E-value |
|---|---|---|---|
| signal peptide | 1 | 18 | — |
| EGF | 59 | 87 | 1.01e−01 |
| EGF_CA | 89 | 128 | 2.13e−9 |
| EGF | 132 | 168 | 2.11e+01 |
| EGF_CA | 169 | 213 | 6.50e−05 |
| EGF_CA | 214 | 254 | 2.85e−1.0 |
| low complexity | 255 | 301 | — |

TABLE 6
Domains Identified by SMART within the
query sequence SCS0008-SV1 of 459 residues
Confidently predicted domains, repeats, motifs and features:

| name | begin | end | E-value |
|---|---|---|---|
| signal peptide | 1 | 9 | — |
| EGF | 57 | 85 | 1.01e−01 |
| EGF_CA | 87 | 126 | 2.13e−09 |
| EGF | 130 | 166 | 2.43e+01 |
| EGF_CA | 167 | 211 | 6.50e−05 |
| EGF_CA | 212 | 252 | 2.85e−10 |
| low complexity | 291 | 305 | — |
| low complexity | 307 | 354 | — |

TABLE 7
Domains identified by SMART within the
query sequence SCS0008-SV2 of 430 residues
Confidently predicted domains, repeats, motifs and features:

| name | begin | end | E-value |
|---|---|---|---|
| signal peptide | 1 | 8 | — |
| EGF | 57 | 85 | 1.01e−01 |
| EGF_CA | 87 | 126 | 2.13e−09 |
| EGF | 130 | 166 | 2.43e+01 |
| EGF_CA | 167 | 211 | 6.50e−05 |
| EGF_CA | 212 | 252 | 2.85e−10 |
| low complexity | 291 | 305 | — |
| low complexity | 307 | 354 | — |

Partial ScanProsite results:

SCS0008:
>PDOC00016 PS00016 RGD Cell attachment sequence [pattern] [Warning: pattern with a high probability of occurrence].
327-329 RGD

SCS0008-SV1:
>PDOC00016 PS00016 RGD Cell attachment sequence [pattern] [Warning: pattern with a high probability of occurrence].
380-382 RGD

SCS0008-SV2:
>PDOC00016 PS00016 RGD Cell attachment sequence [pattern] [Warning: pattern with a high probability of occurrence].
380-382 RGD
   4.2 Description of the Domains

EGF. Epidermal Growth Factor-Like Domain. Interpro Annotation:

A sequence of about thirty to forty amino-acid residues long found in the sequence of epidermal growth factor (EGF) has been shown MEDLINE:, MEDLINE:88196363, MEDLINE:84117505, MEDLINE:91145344, MEDLINE:85063790, MEDLINE: to be present, in a more or less conserved form, in a large number of other, mostly animal proteins. The list of proteins currently known to contain one or more copies of an EGF-like pattern is large and varied. The functional significance of EGF domains in what appear to be unrelated proteins is not yet clear. However, a common feature is that these repeats are found in the extracellular domain of membrane-bound proteins or in proteins known to be secreted (exception: prostaglandin G/H synthase). The EGF domain includes six cysteine residues which have been shown (in EGF) to be involved in disulphide bonds. The main structure is a two-stranded P-sheet followed by a loop to a C-terminal short two-stranded sheet Subdomains between the conserved cysteines vary in length.

GF_CA. Calcium-Binding EGF-Like Domain. Interpro Annotation:

A sequence of about forty amino-acid residues long found in the sequence of epidermal growth factor (EGF) has been shown to be present in a large number of membrane-bound and extracellular, mostly animal proteins (see IPROO0561). Many of these proteins require calcium for their biological function and a calcium-binding site has been found to be located at the N-terminus of some EGF-like domains. Calcium-binding may be crucial for numerous protein-protein interactions. For human coagulation factor IX it has been shown that the calcium-ligands form a pentagonal bipyramid. The first, third and fourth conserved negatively charged or polar residues are side chain ligands. Lafter is possibly hydroxylated (see IPROO0152). A conserved aromatic residue as well as the second conserved negative residue are thought to be involved in stabilizing the calcium-binding site. Like in non-calcium binding EGF-like domains there are six conserved cysteines and the structure of both types is very similar as calcium-binding induces only strictly local structural changes.

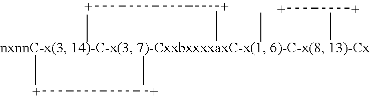

‘In’: negatively charged or polar residue [DEQN]

‘b’: possibly beta-hydroxylated residue [DN]

‘a: aromatic amino acid

‘C’: cysteine, involved in disulfide bond

‘x’: any amino acid.

MAM. Domain in Meprin, AS, Receptor Protein Tyrosine Phosphatase mu (and Others). SMART and Interpro Annotations:

Likely to have an adhesive function. Mutations in the meprin MAM domain affect noncovalent associations within meprin oligomers. In receptor tyrosine phosphatase mu-like molecules the MAM domain is important for homophilic cell-cell interactions. A 170 amino acid domain, the so-called MAM domain, has been recognised in the extracellular region of functionally diverse proteins. These proteins have a modular, receptor-like architecture comprising a signal peptide, an N-terminal extracellular domain, a single transmembrane domain and an intracellular domain. Such proteins include meprin (a cell surface glycoprotein); A5 antigen (a developmentally-regulated cell surface protein); and receptor-like tyrosine protein phosphatase. The MAM domain is thought to have an adhesive function. It contains 4 conserved cysteine residues, which probably form disulphide bridges.

PS00016; RGD

The sequence Arg-Gly-Asp, found in fibronectin, is crucial for its interaction with its cell surface receptor, an integrin. What has been called the ‘RGD’ tripeptide is also found in the sequences of a number of other proteins, where it has been shown to play a role in cell adhesion. These proteins are: some forms of collagens, fibrinogen, vitronectin, von Willebrand factor (VWF), snake disintegrins, and slime mold discoidins. The ‘RGD’ tripeptide is also found in other proteins where it may also, but not always, serve the same purpose.

4.2 Conclusion

Results show that SCS0008, SCSOOOB -SV1 and SCS0008-SV2 display a particular domain organization, being characterized in that they all lack the MAM domain in comparison with the other aligned sequences (see FIG. 21). As described above, the MAM domain has an adhesive function. In addition, Morimura et al. showed that a mutant POEM-Fc molecule without the MAM domain detached from the cell surface and was detected mainly in the culture medium rather than in cell extracts when expressed in COS-7 cells. They conclude by suggesting that the MAM domain plays a significant role for cell surface localization. Interestingly, they point out that the MAM domain family, including meprin, A5 protein, neuropilin-1, neuropilin-2, and receptor protein-tyrosine phosphatases, mediates cell adhesion activities via homo- or heterophilic MAM domain interactions, which supports the hypothesis that the MAM domain is involved in the cell surface binding via protein-protein interaction, and that these molecules could serve as candidate receptor molecules for POEM. SCS0008 and the splice variants SCS0008-SV1 and SCS0008-SV2 could therefore be unable to form homo- or heterophilic MAM domain interactions. As such, the proteins of the invention, possibly being unable to bind to the cell surface, display unique properties. Another possibility is that the sequences of the invention contain a different MAM domain, enabling them to bind to another MAM receptor molecule than the one binding to nephronectin.

In addition, Morimura et al. identified an RGD integrin bindin g motif in POEM and they showed, as well as Brandenberger et al., that POEM is a novel specific ligand molecule for α₈β₁ integrin. The RGD integrin binding motif is present in SCS0008, SCS0008-SV1 and SCS0008-SV2 (see 4.2) suggesting that α₈β₁ integrin could be a specific ligand for these proteins as well. However, Brandenberger et al. showed that the binding site of α₈β₁ integrin appears to be contained in amino acids 382-561 of POEM. As such, SCS0008-SV2, lacking exon 9, might not bind to α₈β₁ integrin.

### Example 5

### Identification of Diseases Related to the Identified Domains

Using SMART and its OMIM curated human diseases associated with missense mutations within domains, human diseases associated with the domains found in SCS0008 or SCS0008-SVS1 or SCS0008-SV2 can be retrieved (tables 8 and 9). Complementary searches were performed with OMIM on the annotated domains. [SMART Annotation]

Diseases Found Associated with Domains

1) SwissProt sequences and OMIM curated human diseases associated with missense mutations within the EGF-like domain.

TABLE 8

| Protein | Disease |
|---|---|
| Coagulation factor IX precursor (EC | (OMIM: 306900): Hemophilia B; |
| 3.4.21.22) (Christmas factor). | Warfarin sensitivity |
| (SRS)(SMART) | |
| Coagulation factor VII precursor (EC | (OMIM: 227500): Factor VII deficiency; |
| 3.4.21.21) (Serum prothrombin conversion | {Myocardial infarction, decreased |
| accelerator) (Eptacog alfa). (SRS)(SMART) | susceptibility to} |
| Fibrillin 2 precursor. (SRS)(SMART) | (OMIM: 121050): Contractural |
| | arachnodactyly, congenital |
| Delta-like protein 3 precursor (Drosophila | (OMIM: 602768): Spondylocostal |
| Delta homolog 3). (SRS)(SMART) | dysostosis, autosomal recessive, 1 |
| | (OMIM: 277300): |
| Vitamin-K-dependent protein C precursor | (OMIM: 176860): Thrombophilia due to |
| (EC 3.4.21.69) (Autoprothrombin IIA) | protein C deficiency; Purpura |
| (Anticoagulant protein C) (Blood coagulation | fulminans, neonatal |
| factor XIV). (SRS)(SMART) | |
| Vitamin K-dependent protein S precursor. | (OMIM: 176880): Protein S deficiency |
| (SRS)(SMART) | |
| Low-density lipoprotein receptor precursor | (OMIM: 143890): |
| (LDL receptor). (SRS)(SMART) | Hypercholesterolemia, familial |
| EGF-containing fibulin-like extracellular | (OMIM: 601548): Doyne honeycomb |
| matrix protein 1 precursor (Fibulin-3) (FIBL- | degeneration of retina |
| 3) (Fibrillin-like protein) (Extracellular protein | (OMIM: 126600): |
| S1-5). (SRS)(SMART) | (OMIM: 126600): Doyne honeycomb |
| | retinal dystrophy |
| Thyroid peroxidase precursor (EC 1.11.1.8) | (OMIM: 274500): Thyroid iodine |
| (TPO). (SRS)(SMART) | peroxidase deficiency; Goiter, |
| | congenital; Hypothyroidism, |
| | congenital |
| Fibrillin 1 precursor. (SRS)(SMART) | (OMIM: 134797): Marfan syndrome |
| | (OMIM: 154700): Shprintzen-Goldberg |
| | syndrome |
| | (OMIM: 182212): Ectopia lentis. familial; |
| | MASS syndrome |
| | (OMIM: 604308): |

Search for other OMIM human diseases maybe associated with the EGF-like domain:

Severe classic or classic or mild variable or neonatal form or atypical Marfan syndrome, aortic aneurysm, marfanoid skeletal syndrome, Weill -Marchesani syndrome, MASP2 deficiency, Factor IX deficiency, megaloblastic anaemia, pseudoachondroplasia, epiphyseal dysplasia, cutis laxa, lissencephaly syndrome (preferably Norman-Roberts type), osteoporosis-pseudoglioma syndrome, osteopetrosis, endosteal hyperostosis, osteoscierosis, high bone mass disorder, dolichostenomelia, micrognathia, congenital kyphoscoliosis, recurrent venous or mesenteric or cerebral venous or arterial thrombosis, superficial thrombophlebitis, Warfarin-induced skin necrosis, retrognathiascoliosis, thoracic lordosis, sphondylolisthesis, lumbosacral dural ectasia, Beals syndrome, coronary heart disease, non-small cell lung cancer, ulcerative colitis or proctitis, endometriosis, chronic obtis, benign prostatic hyperplasia, advanced brain tumor, bladder cancer, duodenal ulcers, metastatic breast cancer, prostate cancer, psoriasis, human uterine leiomyoma, gastric ulcer, discoid lupus erythematosus, lichen planus, colorectal cancer, acute myocardial infarction, advanced malignant glioma, endometrial cancer, malignant gliomas, head and neck or lung cancer, breast cancer, laryngeal and hypopharyngeal carcinoma, ovarian tumours, herpetic comeal ulcers and venous ulcers.

2) SwissProt sequences and OMIM curated human diseases associated with missense mutations within the EGF_CA domain.

TABLE 9

| Protein | Disease |
|---|---|
| Coagulation factor IX precursor (EC | (OMIM: 306900): Hemophilia B; |
| 3.4.21.22) (Christmas factor). (SRS)(SMART) | Warfarin sensitivity |
| E-selectin precursor (Endothelial leukocyte | (OMIM: 131210): {Atherosclerosis, |
| adhesion molecule 1) (ELAM-1) (Leukocyte- | susceptibility to} |
| endothelial cell adhesion molecule 2) | |
| (LECAM2) (CD62E). (SRS)(SMART) | |
| Coagulation factor VII precursor (EC | (OMIM: 227500): Factor VII deficiency; |
| 3.4.21.21) (Serum prothrombin conversion | {Myocardial infarction, decreased |
| accelerator) (Eptacog alfa). (SRS)(SMART) | susceptibility to} |
| Fibrillin 2 precursor. (SRS)(SMART) | (OMIM: 121050): Contractural |
| | arachnodactyly, congenital |
| Delta-like protein 3 precursor (Drosophila | (OMIM: 602768): Spondylocostal |
| Delta homolog 3). (SRS)(SMART) | dysostosis, autosomal recessive, 1 |
| | (OMIM: 277300): |
| Vitamin-K-dependent protein C precursor (EC | (OMIM: 176860): Thrombophilia due |
| 3.4.21.69) (Autoprothrombin IIA) | to protein C deficiency; Purpura |
| (Anticoagulant protein C) (Blood coagulation | fulminans, neonatal |
| factor XIV). (SRS)(SMART) | |
| Vitamin K-dependent protein S precursor. | (OMIM: 176880): Protein S deficiency |
| (SRS)(SMART) | |
| Low-density lipoprotein receptor precursor | (OMIM: 143890): |
| (LDL receptor). (SRS)(SMART) | Hypercholesterolemia, familial |
| EGF-containing fibulin-like extracellular | (OMIM: 601548): Doyne honeycomb |
| matrix protein 1 precursor (Fibulin-3) (FIBL-3) | degeneration of retina |
| (Fibrillin-like protein) (Extracellular protein | (OMIM: 126600): |
| S1-5). (SRS)(SMART) | (OMIM: 126600): Doyne honeycomb |
| | retinal dystrophy |
| Thyroid peroxidase precursor (EC 1.11.1.8) | (OMIM: 274500): Thyroid iodine |
| (TPO). (SRS)(SMART) | peroxidase deficiency; Goiter, |
| | congenital; Hypothyroidism, |
| | congenital |
| Fibrillin 1 precursor. (SRS)(SMART) | (OMIM: 134797): Marfan syndrome |
| | (OMIM: 154700): Shprintzen-Goldberg |
| | syndrome |
| | (OMIM: 182212): Ectopia lentis. |
| | familial; MASS syndrome |
| | (OMIM: 604308): |

Search for other OMIM human diseases maybe associated with the EGF_CA domain:

Severe classic or classic or mild variable or neonatal form or atypical Marfan syndrome, aortic aneurysm, marfanoid skeletal syndrome, Weill-Marchesani syndrome, MASP2 deficiency, Factor IX deficiency, megaloblastic anaemia, pseudoachondroplasia, epiphyseal dysplasia, cutis laxa, lissencephaly syndrome (preferably Norman-Roberts type), osteoporosis-pseudoglioma syndrome, osteopetrosis, endosteal hyperostosis, osteosclerosis, high bone mass disorder, dolichostenomelia, micrognathia, congenital kyphoscoliosis, recurrent venous or mesenteric or cerebral venous or arterial thrombosis, superficial thrombophlebitis, Warfarin-induced skin necrosis, retrognathiascoliosis, thoracic lordosis, sphondylolisthesis, lumbosacral dural ectasia and coronary heart disease.

### Example 6

### Neurological Assays Suitable for Exploration of the Biological Relevance of Proteins Function

A number of neurological assays have been developed by the Applicant and are of use in the investigation of the biological relevance of protein function. Examples of neurological assays that have been developed by the Applicant include four types of assays. These are discussed below.

A. Oligodendrocytes-Based Assays

Oligodendrocytes are responsible for myelin formation in the CNS. In multiple sclerosis they are the first cells attacked and their loss leads to major behavioral impairment In addition to curbing inflammation, enhancing the incomplete remyelinabon of lesions that occurs in MS has been proposed as a therapeutic strategy for MS. Like neurons, mature oligodendrocytes do not divide but the new oligodendrocytes can arise from progenitors. There are very few of these progenitor cells in adult brain and even in embryos the number of progenitor cells is inadequate for HTS.

Oli-neu is a murine cell line obtained by an immortalization of an oligodendrocyte precursor by the t-neu oncogene. They are well studied and accepted as a representative cell line to study young oligodendrocyte biology.

These cells can be used in two types of assays.

One, to identify factors stimulating oligodendrocytes proliferation, and the other to find factors promoting their differentiation. Both events are key in the perspective of helping renewal and repairing demyelinating diseases.

Another possible cell line is the human cell line, MO3-13. MO3-13 results from the fusion of rabdo-myosarcoma cells with adult human oligodendrocytes. However these cells have a reduced ability to differentiate into oligodendrocytes and their proliferating rate is not sufficient to allow a proliferation assay. Nevertheless, they express certain features of oligodendrocytes and their morphology is well adapted to nuclear translocation studies. Therefore this cell line can be used in assays based on nuclear translocation of three transcription factors, respectively NF-kB, Stat-1 and Stat-2. The Jak/Stats transcription pathway is a complex pathway activated by many factors such as IFN α, β, γ, cytokines (e.g. IL-2, IL-6; IL-5) or hormones (e.g. GH, TPO, EPO). The specificity of the response depends on the combination of activated Stats. For example, it is noticeable that IFN-β activates Stat1, 2 and 3 nuclear translocations meanwhile IFN-γ only activates Stat1. In the same way, many cytokines and growth factors induced NF-kB translocation. In these assays the goal is to get a picture of activated pathways for a given protein.

B. Astrocytes-Based Assays

The biology of astrocytes is very complex, but two general states are recognized. In one state called quiescent, astrocytes regulate the metabolic and excitatory level of neurons by pumping glutamate and providing energetic substratum to neurons and oligodendrocytes. In the activated state, astrocytes produce chemokines and cytokines as well as nitric oxide. The first state could be considered as normal healthy while the second state occurs during inflammation, stroke or neurodegenerative diseases. When this activated state persists it should be regarded as a pathological state.

Many factors and many pathways are known to modulate astrocyte activation. In order to identify new factors modulating astrocyte activation U373 cells, a human cell line of astroglioma origin, can be used. NF-kB, c-Jun as well as Stats are signaling molecules known to play pivotal roles in astrocyte activation.

A series of screens based on the nuclear translocation of NF-κB, cJun and Stat1, 2 and 3 can be carried out. Prototypical activators of these pathways are IL-1b, IFN-beta or IFN-gamma. The goal is to identify proteins that could be used as therapeutics in the treatment of CNS diseases.

C. Neurons-Based Assays

Neurons are very complex and diverse cells but they have all in common two things. First they are post-mitouc cells, secondly they are innervating other cells. Their survival is linked to the presence of trophic factors often produced by the innervated target cells. In many neurodegenerative diseases the lost of target innervation leads to cell body atrophy and apoptotic cell death. Therefore identification of trophic factors supplementing target deficiency is very important in treatment of neurodegenerative diseases.

In this perspective a survival assay using NS1 cells, a sub-clone of rat PC12 cells, can be performed. These cells have been used for years and a lot of neurobiology knowledge has been first acquired on these cells before being confirmed on primary neurons including the pathways involved in neuron survival and differentiation (MEK; P13K, CREB). In contrast the N2A cells, a mouse neuroblastoma, are not responding to classical neurotrophic factors but Jun-kinase inhibitors prevent apoptosis induced by serum deprivation. Therefore assays on these two cell lines will help to find different types of “surviving promoting” proteins.

It is important to note that in the previous assays we will identify factors that promote both proliferation and differentiation. In order to identify factors specifically promoting neuronal differentiation, a NS1 differentiation assay based on neurite outgrowth can be used. Promoting axonal or dendritic sprouting in n eurodegenerative diseases could be advantageous for two reasons. It will first help the degenerating neurons to re-grow and re-establish a contact with the target cells. Secondly, it will potentiate the so-called collateral sprouting from healthy fibers, a compensatory phenomenon that delays terminal phases of neurodegenerative such as Parkinson or AD.

D. Endothelial Cells-Based Assays

The blood brain barrier (BBB) between brain and vessels is responsible of differences between cortical spinal fluid and serum compositions. The BBB results from a tight contact between endothelial cells and astrocytes. It maintains an immunotolerant status by preventing leukocytes penetration in brain, and allows the development of two parallels endocrine systems using the same intracellular signaling pathways. However, in many diseases or traumas, the BBB integrity is altered and leukocytes as well as serum proteins enter the brain inducing neuroinflammation. There is no easy in vitro model of BBB, but cultures of primary endothelial cells such as human embryonic umbilical endothelial cells (HWEC) could mimic some aspect of BBB biology. For example, BBB leakiness could be induced by proteins stimulating intracellular calcium release. In the perspective of identifying proteins that modulate BBB integrity, a calcium mobilization assay with or without thrombin can be performed on HUVEC.

### Example 7

### Fibroblast Assays Suitable for Exploration of the Biological Relevance of the Protein Function

A number of fibroblasts assays have been developed by the Applicant and are of use in the investigation of the biological relevance of protein function. Examples of fibroblasts assays that have been developed by the Applicant include eight types of assays.

These are discussed below.

Activation and pathological proliferation of fibroblasts are the key steps leading to a phenotype known as fibrosis. Fibrosis is characterized by the excessive deposition of extracellular matrix, especially collagen. Stromal cells, including fibroblasts, express specific pro- and anti-fibrotic proteins. Keratinocyte growth factor (KGF) is a well-characterized anti-fibrotic molecule. Additionally, oxidative damage and pro-inflammatory stimuli have been proposed to be among major events leading to myofibroblast phenotype and eventually to fibrosis. NF-kB is a mediator of oxidative stress and inflammatory reactions. Based on fibroblast biology, we have developed four cell-based assays, namely fibroblast proliferation, collagen production, NF-kB activation and KGF production assays.

A. Human Fibroblast Proliferation Assay

An activation and pathological proliferation of fibroblasts are the key steps leading to a phenotype known as fibrosis. The assay is based on fluorescence enhancement mediated by CyQUANT GR dye bound to cellular nucleic acids and measures the proliferative responses of human skin-derived fibroblasts to novel proteins and small molecules.

B. Type I Collagen Production by Human Fibroblasts

Fibrosis is characterized by the excessive deposition of extracellular m atrix, especially collagen. Over production of type I collagen is the main manifestation of systemic sclerosis. TGFβ is known to up-regulate production of collagen in vitro and in vivo. We developed cell-based assay in order to test the ability of novel pro-or anti-fibrotic molecules to modulate basal or TGFβ1-stmulated levels of type I collagen production by human skin-derived fibroblasts.

C. Keratinocvte Growth Factor (KGF) Production by Human Fibroblasts

KGF is an important mediator of stroma-to epithelium interactions in many organs (lung, pancreas, kidney, prostate, mammary, gland, uterus) during normal and pathological growth and development. KGF is specifically produced by stromal cells and its receptor is specifically expressed by epithelial cells. It is proposed that KGF might be an important player during pathophysiological reactions in fibrosis and thus can be used as a marker of these reactions. A KGF ELISA assay has been developed and using human lung-derived fibroblasts it has been shown that the KGF production can be significantly up-regulated by IL-1β and TNFα and down-regulated by TGFβ. These cytokines will be used as reference molecules in screening for novel proteins capable to induce KGF production.

D. NF-κB Transcription Activation in Fibroblasts

Oxidative damage and pro-inflammatory stimuli have been proposed to be among major events leading to myofibroblast phenotype and eventually to fibrosis. NF-κB is a mediator of oxidative stress and inflammatory reactions. Swiss 3T3 fibroblasts were generated with a stably integrated NF-κB-SEAP (secreted alkaline phosphatase) construct. NF-κB-SEAP is designed to measure the binding of transcription factors to the ic enhancer allowing a direct measurement of activation of the NF-κB pathway. The SEAP enzyme is secreted into the culture medium, so samples can be collected at various time points to assay for transcription activity without harvesting cells. The Swiss 3T3-NF-κB-SEAP cell line can be used as a cell-based assay to test novel Functional Genomics proteins and is very promising for testing small molecules, especially those with predicted pro-/anti-inflammatory activity.

E. Connective Tissue Growth Factor (CTGF) Promoter Activation/Repression in Fibroblasts

CTGF, a 38-kD cysteine-rich protein, stimulates the production of extracellular matrix elements by fibroblasts. CTGF overexpression has reportedly been found in many fibrotic human tissues, including lung, skin, liver, kidney and blood vessels. In vitro, TGFP activates CTGF gene transcription in human lung fibroblasts. A CTGF promoter-reporter was constructed with secreted alkaline phosphatase (SEAP) as a reporter and Swiss 3T3 fibroblasts were generated with a stably integrated CTGF-SEAP construct Using these fibroblasts it was shown that CTGF promoter is down-regulated by SARP-1, OPG and FSH and up-regulated by TGFβ.

F. KL-6 Production

KL6, originally discovered as a pulmonary adenocarcinoma-related protein and later referred to as MUC-1, is a high-molecular-weight glycoprotein, now classified as Cluster 9 antigen. KL6 is elevated in both sera and BALF of patients with idiopathic pulmonary fibrosis (IPF) and other lung interstitial diseases. In lung tissue from patients suffering from IPF, the majority of cells labelled with KL-6 antibodies are regenerating type II pneumocytes. Two peptides were designed to produce polyclonal antibodies against KL-6. KL-6 ELISA can be used to measure KL-6 production by human lung-derived type II pneumocytes.

G. Neutralization of Apoptosis of L-929 Fibroblasts Treated with Soluble Recombinant TRAIL (TNF-Related Apoptosis-Inducing Ligand)

TRAIL has been shown to be one of the cellular ligands for osteoprotegerin (OPG). This assay can be used to measure the biological activity of OPG.

H. RANKL (Receptor Activator of NF-kB Ligand) Production by Human Fibroblasts

RANKL is another ligand for OPG. This assay can also be used to measure the biological activity of OPG.

### Example 8

### Reproductive Health Assays Suitable for Exploration of the Biological Relevance of proteins Function

A number of reproductive health-related assays have been developed by the Applicant and are of use in the investigation of the biological relevance of SCS0008 protein function. In view of the probable implication of SCS0008 in male infertility (see therapeutic uses), such assays seem of particular relevance. Examples of reproductive health-related assays that have been developed by the Applicant include 18 cell-based assays for reproductive health. These are discussed below.

A. Primary Human Uterine Smooth Muscle Proliferation Assay:

The proliferation of uterine smooth muscle cells is a precursor for development of tumors in uterine fibroid disease in women. In this assay, the goal is to identify proteins that inhibit proliferation of primary human uterine smooth muscle cells.

B. JEG-3 Implantation Assay:

JEG-3 cells are a choriotrophoblastic human cancer cell line used as a model for the blastocyst during implantation. Ishikawa cells are a relatively non-differentated endometrial human cancer cell line that is used as a model for the decidua. JEG-3 cells will “implant” into human decidual tissue. In this assay, a 2-chamber system is used where fluorescently labeled JEG-3 cells invade through a Matrigel-coated porous membrane from an upper chamber into a lower chamber when Ishikawa cells or Ishikawa-conditioned medium are placed into the lower chamber. The cells that migrate are quantified in a plate reader. The goal is to identify proteins that increase invasion of JEG-3 cells for use in aiding implantation in vivo.

C. Osteopontin Bead Assay (Ishikawa Cells):

Ishikawa human endometrial cancer cells are used as a model for implantation. At the time of implantation in the human, various integrins are expressed by the uterine endometrium that is thought to bind to proteins expressed by the blastocyst. Ishikawa cells have been shown in the literature to express avb3, which is the integrin expressed by the uterine endometrium during the “window of implantation”. This integrin is believed to bind the osteopontin expressed by the trophoblast. In this assay, osteopontin-coated fluorescent beads represent the blastocyst, and the Ishikawa cells are primed to accept them for binding by treating them with estradiol. The goal is to identify proteins that increase the ability of the Ishikawa cells to bind the osteopontin-beads as an aid to increase receptivity of the uterine endometrium at the time of implantation.

D. HuF6 Assay:

HuF6 cells are primary human uterine fibroblast cells. These cells can be induced to decidualize by treating them with IL-1β. A marker for decidualization is production of PGE2, which is measured by ELISA. The goal is to identify proteins that increase production of PGE2 by the HuF6 cells as a way of enhancing decidualization during early pregnancy.

E. Endometriosis Assay:

Peritoneal TNFα plays a role in endometriosis by inducing the sloughed endometrial cells from the uterus to adhere to and proliferate on peritoneal mesothelial cells. In this assay, BEND cells are treated with TNFα, which increases their ability to bind fibronectin-coated fluorescent beads as an assay for adherence during endometriosis. The goal is to identify proteins that decrease or inhibit the ability of TNFα to stimulate bead-binding capacity of the cells.

F. Cyclic AMP Assay Using JC410 Porcine Granulose Cells Stably Transfected with hLHR:

In Polycystic Ovary Syndrome, LH from the pituitary is relatively high, and induces androgen output from the ovarian thecal cells. This assay is used to look for an inhibitor of LH signaling which could be used to decrease the action of LH at the ovary during PCOS. The JC410 porcine granulosa cell line is stably transfected with the human LH receptor. Treatment with LH results in CAMP production.

G. Cyclic AMP Assay Using JC410 Porcine Granulose Cells Stably Transfected with hFSHR:

The JC-410 porcine granulosa cell line was stably transfected with the human FSHR. Treatment with FSH stimulates CAMP production, which is measured in this assay. The goal is to identify proteins that enhance FSH action in the granulosa cells.

H. LbetaT2 (Mouse) Pituitary Cells Assay:

The LbetaT2 is an immortalized murine pituitary gonadotroph cell line. Stimulation with Activin alone or with GnRH+Activin results in secretion of FSH (stimulation with GnRH alone results in secretion of LH.) The cells can either be treated with GnRH+Bioscreen proteins to find proteins that act in concert with GnRH to stimulate FSH production, or they can be treated with Bioscreen proteins alone to find a protein that can stimulate FSH secretion like activin alone.

I. Cumulus Expansion Assay:

The cumulus-expansion assay using murine cumulus-oocyte complexes (2/well) has been validated in a 96-well format to assay for proteins that affect oocyte maturation (measured by cumulus expansion). Two 96-well plates can be processed per assay, and 2 assays per week can be performed. If Bioscreen proteins are assayed at only one concentration, all Bioscreen I proteins can be assayed in a month. The read-out may be a yes/no answer for expansion, or image analysis programs may be used to measure expansion in a quantitative manner.

J. RWPE Proliferation Assay:

Benign prostatic hyperplasia is characterized by growth of prostatc epithelium and stroma that is not balanced by apoptosis, resulting in enlargement of the organ. RWPE is a regular human prostatic epithelial cell line that was immortalized with the HPV-18, and may be used in place of primary human prostatic epithelial cells.

K. HT-1080 Fibrosarcoma Invasion Assay:

This assay was developed as a positive cell control for the JEG-3 implantation assay (above). This is a well-established assay as a model for cancer metastasis. Fluorescently-labeled HT-1080 human fibrosarcoma cells are cultured in the upper chamber of a 2-chamber system, and can be stimulated to invade through the porous Matrigel-coated membrane into the bottom chamber where they are quantified. The goal is to identify a protein that inhibits the invasion. The cells are stimulated to invade by adding serum to the bottom chamber and are inhibited with doxycycline.

L. Primary Human Uterine Smooth Muscle Assay:

One of the hallmarks of uterine fibroid disease is collagen deposition by the uterine smooth muscle cells that have become leioymyomas. Primary human uterine smooth muscle cells are stimulated to produce collagen by treatment with TGF β, which is blocked with Rebif. The goal is to discover proteins that inhibit this fibrotic phenotype.

M. Human Leiomvoma Cells Proliferation Assay:

A human leiomyoma cell line may be used as a model for uterine fibroid disease in a proliferation assay. The cells grow very slowly and we are stimulating them to grow at a faster rate by treating them with estradiol and growth factors. The goal is to identify proteins that inhibit estradiol-dependent growth of leiomyoma cells.

N. 937 Migration Assay:

Endometriotic lesions secrete cytokines that recruit immune cells to the peritoneal cavity. These immune cells (especially activated macrophages and T lymphocytes) mediate inflammatory symptoms that are common to endometriosis. RANTES has been shown to be produced by endometrioic stromal cells and is present in the peritoneal fluid. In this assay, U937, a monocytic cell line used as a model for activated macrophages, can be induced by treating the lower level of a 2-chamber culture system to migrate from the upper chamber. If the cells are pre-loaded with fluorescent dye, they can be quantified in the lower chamber. The goal is to identify proteins that inhibit the migration of the U937 cells.

O. JEG3 Human Trophoblast Assay:

The trophoblast of the blastocyst produces HLA-G, a class I HLA molecule that is believed to be important in preventing immunological rejection of the embryo by the mother. During pre-eclampsia, HLA-G levels are low or non-existent, presumably resulting in hallmark symptoms such as poor invasion of the trophoblast into the endometrium and spiral arteries because of maternal immunological interference. The JEG-3 human trophoblast cell line produces HLA-G, which can be increased by treatment with IL-10 or LIF. An ELISA can be used to measure HLA-G production by JEG-3 cells, with the goal being the discovery of other proteins that can increase HLA-G production.

P. Primary Rat Ovarian Dispersate Assay:

Due to the difficulties in measuring appreciable amounts of steroids from the JC410-FSHR/LHR cell lines, an assay using primary cells from whole ovaries taken from immature rats has been developed. Initially, estradiol production from these cultures is measured after treatment with FSH and/or LH. The goal is then to identify proteins that enhance gonadotropin-stimulated steroidogenesis, or proteins that work alone to increase steroidogenesis by these cultures.

Q. Mouse IVF Assay:

In this assay, sperm function, measured by ability to fertilize oocytes, is assayed with the goal of finding proteins that stimulate fertilizing potential of sperm.

R. Primary Human Prostate Stromal Cells Proliferation Assay:

An assay for the epithelial component of BPH has already been described above (see RWPE assay above). This assay uses primary human prostate stromal cells as a model for proliferation of these cells during BPH. The goal is to identify proteins that inhibit proliferation of these cells.

### REFERENCES

Andersen DC and Krummen L, Curr Opin Biotechnol, 13:117-23, 2002.
Baker K N et al., Trends Biotechnol, 20: 149-56, 2002.
Blagoev B and Pandey A, Trends Biochem Sci, 26: 639-41, 2001.
Bock A, Science, 292: 453-4, 2001.
Bunz F, Curr Opin Oncol, 14: 73-8, 2002.
Burgess R R and Thompson N E, Curr Opin Biotechnol, 12: 450-4, 2001.
Chambers S P, Drug Disc Today, 14: 759-765, 2002.
Chu L and Robinson D K, Curr Opin Biotechnol, 13: 304-8, 2001.
Cleland J L et al., Curr Opin Biotechnol, 12: 212-9, 2001.
Coleman R A et al., Drug Discov Today, 6: 1116-1126, 2001.
Constans A, The Scientist, 16(4): 37, 2002.
Davis B G and Robinson M A, Curr Opin Drug Discov Devel, 5: 279-88, 2002.
Dougherty D A, Curr Opin Chem Biol, 4: 645-52, 2000.
Garneft M C, Adv Drug Deliv Rev, 53:171 -216, 2001.
Gavilondo J V and Larrick J W, Biotechniques, 29: 128-136, 2000.
Gendel S M, Ann N Y Acad Sci, 964: 87-98, 2002.
Giddings G, Curr Opin Biotechnol, 12: 450-4, 2001.
Golebiowski A et al., Curr Opin Drug Discov Devel, 4: 428-34, 2001.
Gupta P et al., Drug Discov Today, 7: 569-579, 2002.
Haupt K, Nat Biotechnol, 20 : 884-885, 2002.
Hruby V J and Balse P M, Curr Med Chem, 7: 945-70, 2000.
Johnson D E and Wolfgang G H, Drug Discov Today, 5: 445-454, 2000.
Kane J F, Curr Opin Biotechnol, 6: 494-500, 1995.
Kolb A F, Cloning Stem Cells, 4: 65-80, 2002.
Kuroiwa Y et al., Nat Biotechnol, 20: 889-94, 2002.
Lin Cereghino G P et al., Curr Opin Biotechnol, 13: 329-332, 2001.
Lowe C R et al., J Biochem Biophys Methods, 49: 561 -74, 2001.
Luo B and Prestwich G D, Exp Opin Ther Patents, 11: 1395-1410, 2001.
Mulder N J and Apweiler R, Genome Biol, 3(1):REVIEWS2001, 2002
Nilsson J et al., Protein Expr Purif, 11: 1-16, 1997.
Pearson W R and Miller W, Methods Enzymol, 210: 575 -601, 1992.
Pellois J P et al., Nat Biotechnol, 20: 922-6, 2002.
Pillai O and Panchagnula R, Cur Opin Chem Biol, 5: 447-451, 2001
Rehm B H, Appl Microbiol Biotechnol, 57: 579-92, 2001.
Robinson C R, Nat Biotechnol, 20: 879-880, 2002.
Rogov S I and Nekrasov A N, Protein Eng, 14: 459 -463, 2001.
Schellekens H, Nat Rev Drug Discov, 1: 457-62, 2002
Sheibani N, Prep Biochem Biotechnol, 29: 77-90, 1999.
Stevanovic S, Nat Rev Cancer, 2: 514-20, 2002.
Templin M F et al., Trends Biotechnol, 20: 160-6, 2002.
Tribbick G, J Immunol Methods, 267: 27-35, 2002.
van den Burg B and Eijsink V, Curr Opin Biotechnol, 13: 333 -337, 2002.
van Dijk M A and van de Winkel J G, Curr Opin Chem Biol, 5: 368 -74, 2001.
Villain M et al., Chem Biol, 8: 673-9, 2001.

1-41. (canceled)

42. A composition of matter comprising:
a) an isolated polypeptide having fibrillin-like activity selected from the group consisting of: 1) the amino acid sequence recited in SEQ ID NO: 2; 2) the mature form of the polypeptide whose sequence is recited in SEQ ID NO: 2 (SEQ ID NO:4); 3) active variants of the amino acid sequence of SEQ ID NO: 2, wherein any amino acid specified in the chosen sequence is non-conservatively substituted, provided that no more than 15% of the amino acid residues in the sequence are so changed; and 4) the active fragment, precursor, salt, or derivative of the amino acid sequences given in 1), 2), or 3);
b) an isolated polypeptide that is a naturally occurring allelic variant of the sequence given by SEQ ID NO: 2 or SEQ ID NO:4;
c) an isolated polypeptide that is a naturally occurring allelic variant of the sequence given by SEQ ID NO: 2 or SEQ ID NO:4, wherein the variant is the translation of a single nucleotide polymorphism;
d) a polypeptide as set forth in a) or b) or c), wherein the polypeptide binds specifically an antibody or a binding protein generated against SEQ ID NO: 2 or SEQ ID NO:4 or a fragment thereof;
e) a fusion protein comprising a polypeptide as set forth in a) or b) or c) or d);
f) an antagonist of a polypeptide as set forth in a) or b) or c) or d), wherein said antagonist comprises an amino acid sequence resulting from the non-conservative substitution, the deletion or both the non-conservative substitution and deletion of one or more residues into the corresponding polypeptide;
g) a ligand which binds specifically to a polypeptide a) or b) or c) or d);
h) a polypeptide a) or b) or c) or d) or e), wherein said polypeptides are in the form of active conjugates or complexes with a molecule chosen from radioactive labels, fluorescent labels, biotin, or cytotoxic agents;
i) a peptide mimetic designed on the sequence or the structure or the sequence and structure of a polypeptide as set forth in a) or b) or c) or d);
j) an isolated nucleic acid encoding for an isolated polypeptide selected from the group consisting of:
1) polypeptides as set forth in a) or b) or c) or d);
2) a fusion protein comprising a polypeptide as set forth in a) or b) or c) or d); or
3) an antagonist of a polypeptide as set forth in a) or b) or c) or d), wherein said antagonist comprises an amino acid sequence resulting from the non-conservative substitution, the deletion or both the non-conservative substitution and deletion of one or more residues into the corresponding polypeptide;
k) an isolated nucleic acid sequence consisting of SEQ ID NO: 1; nucleotides 205-2659 of SEQ ID NO: 1; or a complement of either sequence;
l) a purified nucleic acid which:
1) hybridizes under high stringency conditions; or
2) exhibits at least about 85% identity over a stretch of at least about 30 nucleotides with a nucleic acid selected from the group consisting of SEQ ID NO: 1; nucleotides 205-2659 of SEQ ID NO: 1; or a complement of either sequence;
m) a vector comprising a nucleic acid as set forth in j) or k) or 1);
n) a polypeptide encoded by the nucleic acid of j) or k) or 1);
o) a host cell comprising a vector or nucleic acid as set forth in j) or k) or 1) or m);
p) a transgenic animal cell comprising a vector or nucleic acid as set forth in j) or k) or 1) or m) and having enhanced or reduced expression levels of a polypeptide as set forth in a) or b) or c) or d);
q) a transgenic non-human animal that has been transformed to have enhanced or reduced expression levels of a polypeptide as set forth in a) or b) or c) or d);
r) a compound that enhances the expression level of a polypeptide as set forth in a) or b) or c) or d) in a cell or animal; or
s) a compound that reduces the expression level of a polypeptide as set forth in a) or b) or c) or d) in a cell or animal.

43. The composition of matter according to claim 42, wherein said composition of matter comprises a polypeptide, peptide mimetic, nucleic acid, cell, or compound that enhances or reduces the expression of a polypeptide and a pharmaceutically acceptable carrier.

44. The composition of matter according to claim 42, wherein the fusion protein further comprises one or more amino acid sequence selected from the protein sequences: membrane-bound protein, immunoglobulin constant region, multimerization domains, extracellular proteins, signal peptide-containing proteins, or export signal-containing proteins.

45. The composition of matter according to claim 42, wherein the ligand antagonizes or inhibits the fibrillin-like activity of a polypeptide.

46. The composition of matter according to claim 45, wherein the ligand is a monoclonal antibody, a polyclonal antibody, a humanized antibody, an antigen binding fragment, or the extracellular domain of a membrane-bound protein.

47. The composition of matter according to claim 42, wherein said vector comprises a nucleic acid molecule that is operatively linked to expression control sequences allowing expression in prokaryotic or eukaryotic host cells of the encoded polypeptide.

48. A method for determining the activity and/or the presence of a fibrillin-like polypeptide in a sample comprising:
a) providing a protein-containing sample;
b) contacting said sample with a ligand that specifically binds to a polypeptide comprising:
1) an isolated polypeptide having fibrillin-like activity selected from the group consisting of: i) the amino acid sequence recited in SEQ ID NO: 2; ii) the mature form of the polypeptide whose sequence is recited in SEQ ID NO: 2 (SEQ ID NO:4); iii) active variants of the amino acid sequence of SEQ ID NO: 2, wherein any amino acid specified in the chosen sequence is non-conservatively substituted, provided that no more than 15% of the amino acid residues in the sequence are so changed; and iv) the active fragment, precursor, salt, or derivative of the amino acid sequences given in i), ii), or iii);
2) an isolated polypeptide that is a naturally occurring allelic variant of the sequence given by SEQ ID NO: 2 or SEQ ID NO:4;
3) an isolated polypeptide that is a naturally occurring allelic variant of the sequence given by SEQ ID NO: 2 or SEQ ID NO:4, wherein the variant is the translation of a single nucleotide polymorphism; or
4) a polypeptide as set forth in a) or b) or c), wherein the polypeptide binds specifically an antibody or a binding protein generated against SEQ ID NO: 2 or SEQ ID NO:4 or a fragment thereof; and
c) determining the presence or said ligand bound to said polypeptide.

49. A method of using the composition of claim 42 for a) producing cells capable of expressing a polypeptide; b) making a polypeptide; c) the treatment of a disease; d) the preparation of pharmaceutical compositions; e) the screening candidate compounds; f) identifying a candidate compound; or g) determining the presence or the amount of a transcript or of a nucleic acid.

50. The method according to claim 49, wherein said method comprises genetically engineering cells with a vector or a nucleic acid comprising:
a) an isolated nucleic acid encoding for an isolated polypeptide selected from the group consisting of:
1) an isolated polypeptide having fibrillin-like activity selected from the group consisting of: i) the amino acid sequence recited in SEQ ID NO: 2; ii) the mature form of the polypeptide whose sequence is recited in SEQ ID NO: 2 (SEQ ID NO:4); iii) active variants of the amino acid sequence of SEQ ID NO: 2, wherein any amino acid specified in the chosen sequence is non-conservatively substituted, provided that no more than 15% of the amino acid residues in the sequence are so changed; and iv) the active fragment, precursor, salt, or derivative of the amino acid sequences given in i), ii), or iii);
2) an isolated polypeptide that is a naturally occurring allelic variant of the sequence given by SEQ ID NO: 2 or SEQ ID NO:4;
3) an isolated polypeptide that is a naturally occurring allelic variant of the sequence given by SEQ ID NO: 2 or SEQ ID NO:4, wherein the variant is the translation of a single nucleotide polymorphism;
4) a polypeptide as set forth in 1) or 2) or 3), wherein the polypeptide binds specifically an antibody or a binding protein generated against SEQ ID NO: 2 or SEQ ID NO:4 or a fragment thereof; or
5) a fusion protein comprising a polypeptide as set forth in 1) or 2) or 3) or 4);
b) an isolated nucleic acid sequence consisting of SEQ ID NO: 1; nucleotides 205-2659 of SEQ ID NO: 1; or a complement of either sequence;
c) a purified nucleic acid which:
1) hybridizes under high stringency conditions; or
2) exhibits at least about 85% identity over a stretch of at least about 30 nucleotides with a nucleic acid selected from the group consisting of SEQ ID NO: 1; nucleotides 205-2659 or SEQ ID NO: 1; or a complement of either sequence; or
d) a vector comprising a nucleic acid as set forth in a) or b) or c).

51. The method according to claim 49, wherein said method comprises a method for making a polypeptide comprising culturing a transformed host cell under conditions in which the nucleic acid or vector is expressed, and recovering the polypeptide encoded by said nucleic acid or vector from the culture.

52. The method according to claim 49, wherein said method comprises the treatment of a disease needing an increase in the fibrillin-like activity that comprises the administration of a therapeutically effective amount of a polypeptide, a peptide mimetic, a nucleic acid, a cell, or a compound as set forth in claim 42.

53. The method according to claim 49, wherein said method of treatment comprises the administration of a therapeutically effective amount of an antagonist, a ligand, or of a compound as set forth in claim 42.

54. The method according to claim 49, wherein said method for screening candidate compounds effective to treat a disease related to the fibrillin-like polypeptides comprises:
a) contacting a cell, a transgenic animal cell, or a transgenic non-human animal having enhanced or reduced expression levels of the fibrillin-like polypeptide, with a candidate compound; and
b) determining the effect of the compound on the animal or on the cell.

55. The method according to claim 49, wherein said method for identifying a candidate compound as an antagonist/inhibitor or agonist/activator of a fibrillin-like polypeptide comprises:
a) contacting said polypeptide, said compound, and a mammalian cell or a mammalian cell membrane capable of binding the polypeptide; and
b) measuring whether the molecule blocks or enhances the interaction of the polypeptide, or the response that results from such interaction, with the mammalian cell or the mammalian cell membrane.

56. The method according to claim 49, wherein said method for determining the presence or the amount of transcript or of a nucleic acid encoding a fibrillin-like polypeptide comprises:
a) providing a nucleic acids-containing sample;
b) contacting said sample with a nucleic acid as set forth in claim 42; and
c) determining the hybridization of said nucleic acid with a nucleic acid into the sample.


SEQUENCE LISTING (18 sequences)

SEQ ID NO: 1 - DNA, 1700 bp, homo sapiens
  CDS (205)..(1425)
gcgggagggggctccgggcgccgcgcagcagacctgctccggccgcgcgcctcgccgctgtcctccgggagcggcagcagtagcccgggcggcgagggctgggggttcctcgagactctcagaggggcgcctcccatcggcgcccaccaccccaacctgttcctcgcgcgccactgcgctgcgccccaggacccgctgcccaacatggattttctcctggcgctggtgctggtatcctcgctctacctgcaggcggccgccgagttcgacgggaggtggcccaggcaaatagtgtcatcgattggcctatgtcgttatggtgggaggattgactgctgctggggctgggctcgccagtcttggggacagtgtcagcctgtgtgccaaccacgatgcaaacatggtgaatgtatcgggccaaacaagtgcaagtgtcatcctggttatgctggaaaaacctgtaatcaagatctaaatgagtgtggcctgaagccccggccctgtaagcacaggtgcatgaacacttacggcagctacaagtgctactgtctcaacggatatatgctcatgccggatggttcctgctcaagtgccctgacctgctccatggcaaactgtcagtatggctgtgatgttgttaaaggacaaatacggtgccagtgcccatcccctggcctgcagctggctcctgatgggaggacctgtgtagatgttgatgaatgtgctacaggaagagcctcctgccctagatttaggcaatgtgtcaacacttttgggagctacatctgcaagtgtcataaaggcttcgatctcatgtatattggaggcaaatatcaatgtcatgacatagacgaatgctcacttggtcagtatcagtgcagcagctttgctcgatgttataacgtacgtgggtcctacaagtgcaaatgtaaagaaggataccagggtgatggactgacttgtgtgcctacttctaagccaacaacaagacctacaccaaagccaacaccaattcctactccaccaccaccaccacccctgccaacagagctcagaacacctctaccacctacaaccccagaaaggccaaccaccggactgacaactatagcaccagctgccagtacacctccaggagggattacagttgacaacagggtacagacagaccctcagaaacccagaggagatgtgttcattccacggcaaccttcaaatgacttgtttgaaatatttgaaatagaaagaggagtcagtgcagacgatgaagcaaaggatgatccaggtgttctggtacacagttgtaattttgaccatggactttgtggatggatcagggagaaagacaatgacttgcactgggaaccaatcagggacccagcagacaattgtagctgtctgcatgcaggtggacttgacttgtagcaactggagacaaggtgtagttctccatgtgtctgagattccagaactagactcaattgtggttccagatcaaatcagcctgcagagaatggctgttccctggtgcacatggagttgagcggagcagcacacttgggcacaggggttggccgtcagctcactgtcctgctgccaccctccaccacctcagaggctgctgtaactttgagaaaataacctttgaacctcagtctctacaatgcttaaataaagagaacattcaattagagaaaata

SEQ ID NO: 2 - protein, 406 aa, homo sapiens
MDFLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLQLAPDGRTCVDVDECATGRASCPRFRQCVNTFGSYICKCHKGFDLMYIGGKYQCHDIDECSLGQYQCSSFARCYNVRGSYKCKCKEGYQGDGLTCVPTSKPTTRPTPKPTPIPTPPPPPPLPTELRTPLPPTTPERPTTGLTTIAPAASTPPGGITVDNRVQTDPQKPRGDVFIPRQPSNDLFEIFEIERGVSADDEAKDDPGVLVHSCNFDHGLCGWIREKDNDLHWEPIRDPADNCSCLHAGGLDL

SEQ ID NO: 3 - protein, 404 aa, homo sapiens
FLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLQLAPDGRTCVDVDECATGRASCPRFRQCVNTFGSYICKCHKGFDLMYIGGKYQCHDIDECSLGQYQCSSFARCYNVRGSYKCKCKEGYQGDGLTCVPTSKPTTRPTPKPTPIPTPPPPPPLPTELRTPLPPTTPERPTTGLTTIAPAASTPPGGITVDNRVQTDPQKPRGDVFIPRQPSNDLFEIFEIERGVSADDEAKDDPGVLVHSCNFDHGLCGWIREKDNDLHWEPIRDPADNCSCLHAGGLDL

SEQ ID NO: 4 - protein, 412 aa, homo sapiens
MDFLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLQLAPDGRTCVDVDECATGRASCPRFRQCVNTFGSYICKCHKGFDLMYIGGKYQCHDIDECSLGQYQCSSFARCYNVRGSYKCKCKEGYQGDGLTCVPTSKPTTRPTPKPTPIPTPPPPPPLPTELRTPLPPTTPERPTTGLTTIAPAASTPPGGITVDNRVQTDPQKPRGDVFIPRQPSNDLFEIFEIERGVSADDEAKDDPGVLVHSCNFDHGLCGWIREKDNDLHWEPIRDPADNCSCLHAGGLDLHHHHHH

SEQ ID NO: 5 - DNA, 1590 bp, homo sapiens
  CDS (205)..(1590)
gcgggagggggctccgggcgccgcgcagcagacctgctccggccgcgcgcctcgccgctgtcctccgggagcggcagcagtagcccgggcggcgagggctgggggttcctcgagactctcagaggggcgcctcccatcggcgcccaccaccccaacctgttcctcgcgcgccactgcgctgcgccccaggacccgctgcccaacatggattttctcctggcgctggtgctggtatcctcgctctacctgcaggcggccgccgagttcgacgggaggtggcccaggcaaatagtgtcatcgattggcctatgtcgttatggtgggaggattgactgctgctggggctgggctcgccagtcttggggacagtgtcagcctgtgtgccaaccacgatgcaaacatggtgaatgtatcgggccaaacaagtgcaagtgtcatcctggttatgctggaaaaacctgtaatcaagatctaaatgagtgtggcctgaagccccggccctgtaagcacaggtgcatgaacacttacggcagctacaagtgctactgtctcaacggatatatgctcatgccggatggttcctgctcaagtgccctgacctgctccatggcaaactgtcagtatggctgtgatgttgttaaaggacaaatacggtgccagtgcccatcccctggcctgcacctggctcctgatgggaggacctgtgtagatgttgatgaatgtgctacaggaagagcctcctgccctagatttaggcaatgtgtcaacacttttgggagctacatctgcaagtgtcataaaggcttcgatctcatgtatattggaggcaaatatcaatgtcatgacatagacgaatgctcacttggtcagtatcagtgcagcagctttgctcgatgttataacgtacgtgggtcctacaagtgcaaatgtaaagaaggataccagggtgatggactgacttgtgtgtatatcccaaaagttatgattgaaccttcaggtccaattcatgtaccaaagggaaatggtaccattttaaagggtgacacaggaaataataattggattcctgatgttggaagtacttggtggcctccgaagacaccatatattcctcctatcattaccaacaggcctacttctaagccaacaacaagacctacaccaaagccaacaccaattcctactccaccaccaccaccacccctgccaacagagctcagaacacctctaccacctacaaccccagaaaggccaaccaccggactgacaactatagcaccagctgccagtacacctccaggagggattacagttgacaacagggtacagacagaccctcagaaacccagaggagatgtgttcattccacggcaaccttcaaatgacttgtttgaaatatttgaaatagaaagaggagtcagtgcagacgatgaagcaaaggatgatccaggtgttctggtacacagttgtaattttgaccatggactttgtggatggatcagggagaaagacaatgacttgcactgggaaccaatcagggacccagcagacaattgtagctgtctgcatgcaggtggacttgacttgtag

SEQ ID NO: 6 - protein, 461 aa, homo sapiens
MDFLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLHLAPDGRTCVDVDECATGRASCPRFRQCVNTFGSYICKCHKGFDLMYIGGKYQCHDIDECSLGQYQCSSFARCYNVRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFIPRQPSNDLFEIFEIERGVSADDEAKDDPGVLVHSCNFDHGLCGWIREKDNDLHWEPIRDPADNCSCLHAGGLDL

SEQ ID NO: 7 - protein, 461 aa, homo sapiens
MDFLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLQLAPDGRTCVDVDECATGRASCPRFRQCVNTFGSYICKCHKGFDLMYIGGKYQCHDIDECSLGQYQCSSFARCYNVRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFIPRQPSNDLFEIFEIERGVSADDEAKDDPGVLVHSCNFDHGLCGWIREKDNDLHWEPIRDPADNCSCLHAGGLDL

SEQ ID NO: 8 - protein, 459 aa, homo sapiens
FLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLHLAPDGRTCVDVDECATGRASCPRFRQCVNTFGSYICKCHKGFDLMYIGGKYQCHDIDECSLGQYQCSSFARCYNVRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFIPRQPSNDLFEIFEIERGVSADDEAKDDPGVLVHSCNFDHGLCGWIREKDNDLHWEPIRDPADNCSCLHAGGLDL

SEQ ID NO: 9 - protein, 459 aa, homo sapiens
FLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLQLAPDGRTCVDVDECATGRASCPRFRQCVNTFGSYICKCHKGFDLMYIGGKYQCHDIDECSLGQYQCSSFARCYNVRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFIPRQPSNDLFEIFEIERGVSADDEAKDDPGVLVHSCNFDHGLCGWIREKDNDLHWEPIRDPADNCSCLHAGGLDL

SEQ ID NO: 10 - protein, 467 aa, homo sapiens
MDFLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLHLAPDGRTCVDVDECATGRASCPRFRQCVNTFGSYICKCHKGFDLMYIGGKYQCHDIDECSLGQYQCSSFARCYNVRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFIPRQPSNDLFEIFEIERGVSADDEAKDDPGVLVHSCNFDHGLCGWIREKDNDLHWEPIRDPADNCSCLHAGGLDLHHHHHH

SEQ ID NO: 11 - protein, 467 aa, homo sapiens
MDFLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLQLAPDGRTCVDVDECATGRASCPRFRQCVNTFGSYICKCHKGFDLMYIGGKYQCHDIDECSLGQYQCSSFARCYNVRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFIPRQPSNDLFEIFEIERGVSADDEAKDDPGVLVHSCNFDHGLCGWIREKDNDLHWEPIRDPADNCSCLHAGGLDLHHHHHH

SEQ ID NO: 12 - DNA, 1503 bp, homo sapiens
  CDS (205)..(1503)
gcgggagggggctccgggcgccgcgcagcagacctgctccggccgcgcgcctcgccgctgtcctccgggagcggcagcagtagcccgggcggcgagggctgggggttcctcgagactctcagaggggcgcctcccatcggcgcccaccaccccaacctgttcctcgcgcgccactgcgctgcgccccaggacccgctgcccaacatggatttgctcctggcgctggtgctggtatcctcgctctacctgcaggcggccgccgagttcgacgggaggtggcccaggcaaatagtgtcatcgattggcctatgtcgttatggtgggaggattgactgctgctggggctgggctcgccagtcttggggacagtgtcagcctgtgtgccaaccacgatgcaaacatggtgaatgtatcgggccaaacaagtgcaagtgtcatcctggttatgctggaaaaacctgtaatcaagatctaaatgagtgtggcctgaagccccggccctgtaagcacaggtgcatgaacacttacggcagctacaagtgctactgtctcaacggatatatgctcatgccggatggttcctgctcaagtgccctgacctgctccatggcaaactgtcagtatggctgtgatgttgttaaaggacaaatacggtgccagtgcccatcccctggcctgcacctggctcctgatgggaggacctgtgtagatgttgatgaatgtgctacaggaagagcctcctgccctagatttaggcaatgtgtcaacacttttgggagctacatctgcaagtgtcataaaggcttcgatctcatgtatattggaggcaaatatcaatgtcatgacatagacgaatgctcacttggtcagtatcagtgcagcagctttgctcgatgttataacgtacgtgggtcctacaagtgcaaatgtaaagaaggataccagggtgatggactgacttgtgtgtatatcccaaaagttatgattgaaccttcaggtccaattcatgtaccaaagggaaatggtaccattttaaagggtgacacaggaaataataattggattcctgatgttggaagtacttggtggcctccgaagacaccatatattcctcctatcattaccaacaggcctacttctaagccaacaacaagacctacaccaaagccaacaccaattcctactccaccaccaccaccacccctgccaacagagctcagaacacctctaccacctacaaccccagaaaggccaaccaccggactgacaactatagcaccagctgccagtacacctccaggagggattacagttgacaacagggtacagacagaccctcagaaacccagaggagatgtgttcagtgttctggtacacagttgtaattttgaccatggactttgtggatggatcagggagaaagacaatgacttgcactgggaaccaatcagggacccagcagacaattgtagctgtctgcatgcaggtggacttgacttgtag

SEQ ID NO: 13 - protein, 432 aa, homo sapiens
MDLLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLHLAPDGRTCVDVDECATGRASCPRFRQCVNTFGSYICKCHKGFDLMYIGGKYQCHDIDECSLGQYQCSSFARCYNVRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFSVLVHSCNFDHGLCGWIREKDNDLHWEPIRDPADNCSCLHAGGLDL

SEQ ID NO: 14 - protein, 432 aa, homo sapiens
MDFLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLQLAPDGRTCVDVDECATGRASCPRFRQCVNTFGSYICKCHKGFDLMYIGGKYQCHDIDECSLGQYQCSSFARCYNVRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFSVLVHSCNFDHGLCGWIREKDNDLHWEPIRDPADNCSCLHAGGLDL

SEQ ID NO: 15 - protein, 430 aa, homo sapiens
LLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLHLAPDGRTCVDVDECATGRASCPRFRQCVNTFGSYICKCHKGFDLMYIGGKYQCHDIDECSLGQYQCSSFARCYNVRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFSVLVHSCNFDHGLCGWIREKDNDLHWEPIRDPADNCSCLHAGGLDL

SEQ ID NO: 16 - protein, 430 aa, homo sapiens
FLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLQLAPDGRTCVDVDECATGRASCPRFRQCVNTFGSYICKCHKGFDLMYIGGKYQCHDIDECSLGQYQCSSFARCYNVRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFSVLVHSCNFDHGLCGWIREKDNDLHWEPIRDPADNCSCLHAGGLDL

SEQ ID NO: 17 - protein, 438 aa, homo sapiens
MDLLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLHLAPDGRTCVDVDECATGRASCPRFRQCVNTFGSYICKCHKGFDLMYIGGKYQCHDIDECSLGQYQCSSFARCYNVRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFSVLVHSCNFDHGLCGWIREKDNDLHWEPIRDPADNCSCLHAGGLDLHHHHHH

SEQ ID NO: 18 - protein, 438 aa, homo sapiens
MDFLLALVLVSSLYLQAAAEFDGRWPRQIVSSIGLCRYGGRIDCCWGWARQSWGQCQPVCQPRCKHGECIGPNKCKCHPGYAGKTCNQDLNECGLKPRPCKHRCMNTYGSYKCYCLNGYMLMPDGSCSSALTCSMANCQYGCDVVKGQIRCQCPSPGLQLAPDGRTCVDVDECATGRASCPRFRQCVNTFGSYICKCHKGFDLMYIGGKYQCHDIDECSLGQYQCSSFARCYNVRGSYKCKCKEGYQGDGLTCVYIPKVMIEPSGPIHVPKGNGTILKGDTGNNNWIPDVGSTWWPPKTPYIPPIITNRPTSKPTTRPTPKPTPIPTPPPPPPLPTELRTPLPPTTPERPTTGLTTIAPAASTPPGGITVDNRVQTDPQKPRGDVFSVLVHSCNFDHGLCGWIREKDNDLHWEPIRDPADNCSCLHAGGLDLHHHHHH